(19) United States
(12) Patent Application Publication
(10) Pub. No.: US 2022/0324898 A1
(43) Pub. Date: Oct. 13, 2022

(54) PROSTATE SPECIFIC MEMBRANE ANTIGEN (PSMA) LIGANDS COMPRISING AN AMYLASE CLEAVABLE LINKER

(71) Applicants: Deutsches Krebsforschungszentrum, Heidelberg (DE); Ruprecht-Karls-Universität Heldelberg, Heidelberg (DE)

(72) Inventors: Matthias Eder, Simonswald (DE); klaus Kopka, Dresden (DE); Martin Schäfer, Neckarsteinach (DE); Ulrike Bauder-Wuest, Schriesheim (DE); Uwe Haberkrn, Schwetzingen (DE); Hans-Christian Kliem, Heppenheim (DE); Walter Mier, Bensheim (DE); Thomas Lindner, Schriesheim (DE)

(21) Appl. No.: 17/431,248

(22) PCT Filed: Feb. 14, 2020
(86) PCT No.: PCT/EP2020/053891

§ 371 Date: Aug. 16, 2021

(30) Foreign Application Priority Data
Feb. 14, 2019 (EP) 19157216.3
May 31, 2019 (EP) 19177667.3

(51) Int. Cl. C07H 17/04; G01N 33/574; A61K 51/04

(52) U.S. Cl. CPC C07H 17/04; G01N 33/57434; A61K 51/0491

(57) ABSTRACT

In particular, the present invention relates to a PSMA binding ligand comprising an oligosaccharide building block which comprises a bond being cleavable by alpha-amylase.

Typically, this PSMA binding ligand further comprises a PSMA binding motif Q and a chelator residue A, wherein the PSMA binding motif Q and the chelator residue A are preferably linked via at least one linker L^{AQ} comprising the oligosaccharide building block, the PSMA binding ligand thus preferably having the structure (I)

A-L^{AQ}-Q

or a pharmaceutically acceptable salt or solvate thereof.

### CROSS-REFERENCE TO RELATED APPLICATIONS

This application is a U.S. National Phase of International PCT Application No. PCT/EP2020/053891 filed on Feb. 14, 2020, which claims priority to European Patent Application No. 19157216.3 filed on Feb. 14, 2019 and European Patent Application No. 19177667.3 filed on May 31, 2019, the contents of each application are incorporated herein by reference in their entirety.

### FIELD OF THE INVENTION

The present invention generally relates to the field of radiopharmaceuticals and their use in nuclear medicine as tracers, imaging agents and for the treatment of various disease states of PSMA-expressing cancers, especially prostate cancer, and metastases thereof.

### RELATED ART

Prostate cancer (PCa) is the leading cancer in the US and European population. At least 1-2 million men in the western hemisphere suffer from prostate cancer and it is estimated that the disease will strike one in six men between the ages of 55 and 85. There are more than 300,000 new cases of prostate cancer diagnosed each year in USA. The mortality from the disease is second only to lung cancer. Currently, imaging methods with high resolution of the anatomy, such as computed tomography (CT), magnetic resonance (MR) imaging and ultrasound, predominate for clinical imaging of prostate cancer. An estimated annual $2 billion is currently spent worldwide on surgical, radiation, drug therapy and minimally invasive treatments. However, there is presently no effective therapy for relapsing, metastatic, androgen-independent prostate cancer.

A variety of experimental low molecular weight PCa imaging agents are currently being pursued clinically, including radiolabeled choline analogs [¹⁸F]fluorodihydrotestosterone ([¹⁸F]FDHT), anti-1-amino-3-[¹⁸F]fluorocycIobutyl-1-carboxylic acid (anti[¹⁸F]F-FACBC, [¹¹C]acetate and 1-(2-deoxy-2-[¹⁸F]flouro-L-arabinofuranosyl)-5-methyluracil (—[¹⁸F]FMAU) (Scher, B.; et al. Eur J Nucl Med Mol Imaging 2007, 34, 45-53; Rinnab, L; et al. BJU Int 2007, 100, 786, 793; Reske, S. N.; et al. J Nucl Med 2006, 47, 1249-1254; Zophel, K.; Kotzerke, J. Eur J Nucl Med Mol Imaging 2004, 31, 756-759; Vees, H.; et al. BJU Int 2007, 99, 1415-1420; Larson, S. M.; et al. J Nucl Med 2004, 45, 366-373; Schuster, D. M.; et al. J Nucl Med 2007, 48, 56-63; Tehrani, O. S.; et al. J Nucl Med 2007, 48, 1436-1441). Each operates by a different mechanism and has certain advantages, e.g., low urinary excretion for [¹¹C]choline, and disadvantages, such as the short physical half-life of positron-emitting radionuclides.

It is well known that tumors may express unique proteins associated with their malignant phenotype or may over-express normal constituent proteins in greater number than normal cells. The expression of distinct proteins on the surface of tumor cells offers the opportunity to diagnose and characterize disease by probing the phenotypic identity and biochemical composition and activity of the tumor. Radioactive molecules that selectively bind to specific tumor cell surface proteins provide an attractive route for imaging and treating tumors under non-invasive conditions. A promising new series of low molecular weight imaging agents targets the prostate-specific membrane antigen (PSMA) (Mease R. C. et al. Clin Cancer Res. 2008, 14, 3036-3043; Foss, C. A.; et al. Clin Cancer Res 2005, 11, 4022-4028; Pomper, M. G.; et al. Mol Imaging 2002, 1, 96-101; Zhou, J.; et al. Nat Rev Drug Discov 2005, 4, 015-1026; WO 2013/022797).

PSMA is a trans-membrane, 750 amino acid type II glycoprotein that has abundant and restricted expression on the surface of PCa, particularly in androgen-independent, advanced and metastatic disease (Schulke, N.; et al. Proc Natl Acad Sci USA 2003, 100, 12590-12595). The latter is important since almost all PCa become androgen independent over the time. PSMA possesses the criteria of a promising target for therapy (Schulke, N.; et al. Proc. Natl. Acad. Sci. USA 2003, 100, 12590-12595). The PSMA gene is located on the short arm of chromosome 11 and functions both as a folate hydrolase and neuropeptidase. It has neuropeptidase function that is equivalent to glutamate carboxypeptidase II (GCPII), which is referred to as the “brain PSMA”, and may modulate glutamatergic transmission by cleaving/V-acetylaspartylglutamate (NAAG) to N-acetylaspartate (NAA) and glutamate (Nan, F.; et al. J Med Chem 2000, 43, 772-774). There are up to 10⁶ PSMA molecules per cancer cell, further suggesting it as an ideal target for imaging and therapy with radionuclide-based techniques (Tasch, J.; et al. Crit Rev Immunol 2001, 21, 249-261).

The radio-immunoconjugate of the anti-PSMA monoclonal antibody (mAb) 7E11, known as the PROSTASCINT® scan, is currently being used to diagnose prostate cancer metastasis and recurrence. However, this agent tends to produce images that are challenging to interpret (Lange, P. H. PROSTASCINT scan for staging prostate cancer. Urology 2001, 57, 402-406; Haseman, M. K.; et al. Cancer Biother Radiopharm 2000, 15, 131-140; Rosenthal, S. A.; et al. Tech Urol 2001, 7, 27-37). More recently, monoclonal antibodies have been developed that bind to the extracellular domain of PSMA and have been radiolabeled and shown to accumulate in PSMA-positive prostate tumor models in animals. However, diagnosis and tumor detection using monoclonal antibodies has been limited by the low permeability of the monoclonal antibody in solid tumors.

The selective targeting of cancer cells with radiopharmaceuticals, either for imaging or therapeutic purposes is challenging. A variety of radionuclides are known to be useful for radio-imaging or cancer radiotherapy, including ¹¹¹In, ⁹⁰Y, ⁶⁸Ga, ¹⁷⁷Lu, ^{99m}TC, ¹²³I, and ¹³¹I. Recently it has been shown that some compounds containing a glutamate-urea-glutamate (GUG) or a glutamate-urea-lysine (GUL) recognition element linked to a radionuclide-ligand conjugate exhibit high affinity for PSMA.

In WO 2015/055318 new imaging agents with improved tumor targeting properties and pharmacokinetics were described. These compounds comprise a motif specifically binding to cell membranes of cancerous cells, wherein said motif comprises a prostate-specific membrane antigen (PSMA), that is the above mentioned glutamate-urea-lysine motif. The preferred molecules described in WO 2015/055318 further comprise a linker which binds via an amide bond to a carboxylic acid group of DOTA as chelator. Some of these compounds have been shown to be promising agents for the specific targeting of prostate tumors. The compounds were labeled with ¹⁷⁷Lu (for therapy purposes) or ⁶⁸Ga (for diagnostic purposes) and allow for visualization and targeting of prostate cancer for radiotherapy purposes.

However, in therapeutic applications of radioactively labeled PSMA inhibitors, organs with physiological PSMA expression turned out to be dose limiting and thus minimize the therapeutic success. In particular, the high renal and salivary gland uptake of the radioactively labeled PSMA inhibitor substances is noticeable, which, in the case of a therapeutic application, gives rise to considerable side effects. Attempts to improve the kidney uptake of PSMA inhibitors has led to the development of PSMA-617 [Benesova, M., et al. (2016) J Med Chem 59, 1761-75], a compound which is already used clinically with 177Lu or 225Ac for endoradiotherapy of prostate cancer. However, a reduction in salivary and lacrimal gland uptake has not yet been achieved and is still described as critical and dose-limiting in early clinical work. In a first-in-man study with 225Ac-PSMA-617, two patients with extremely advanced and end-stage disease showed complete remission. In both patients the PSA value fell below the detectability limit. Accompanying diagnostic recordings with 68Ga-PSMA-11 confirmed a complete response.

As already mentioned above, the strong accumulation of PSMA ligands in the salivary and lacrimal glands, which is described in numerous papers leads to considerable side effects. The salivary and lacrimal glands are severely and partially irreversibly damaged, in particular during alpha therapy with 225Ac. The resulting xerostomia for example represents a dose-limiting side effect.

Thus, there is still the need for improved PSMA ligands which provide advantageous options for the detection, treatment and management of PSMA-expressing cancers, in particular prostate cancer, and which preferably show less side effects on the salivary glands and/or lacrimal glands.

### SUMMARY OF THE INVENTION

The solution of said object is achieved by providing the embodiments characterized in the claims. The inventors found new PSMA binding ligands which are useful and advantageous radiopharmaceuticals and which can be used in nuclear medicine as tracers, imaging agents and for the treatment of various disease states of PSMA-expressing cancers, in particular prostate cancer. These compounds are described in more detail below:

In particular, the present invention relates to a PSMA binding ligand or a pharmaceutically acceptable salt or solvate thereof, the PSMA binding ligand comprising an oligosaccharide building block which comprises a bond being cleavable by alpha-amylase.

Typically, this PSMA binding ligand further comprises a PSMA binding motif Q and a chelator residue A, wherein the PSMA binding motif Q and the chelator residue A are preferably linked via at least one linker L^{AQ} comprising the oligosaccharide building block, the PSMA binding ligand thus preferably having the structure (I)

A-L^{AQ}-Q.

Further, the present invention relates t to a PSMA binding ligand or a pharmaceutically acceptable salt or solvate thereof, the PSMA binding ligand having a structure according to formula (Ia)

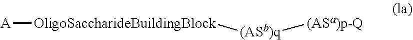

wherein Q is a PSMA binding motif, A is a chelator residue, AS^{a} and AS^{b} are amino acid building blocks and q is an integer of from 0-4, preferably 0 to 3, more preferably 1, and p is an integer of from 0-3, preferably of from 1 to 3.

Further, the present invention relates to a PSMA binding ligand of formula (II)

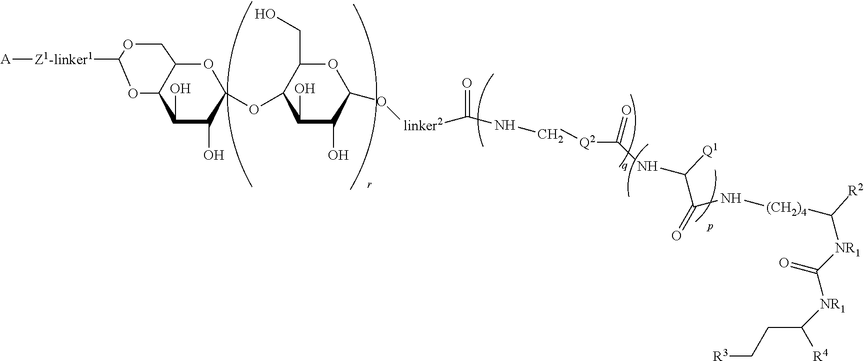

or a pharmaceutically acceptable salt or solvate thereof, wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂, Q¹ is selected from the group consisting of alkylaryl, arylalkyl, aryl, alkylheteroaryl, heteroarylalkyl and heteroaryl, Q² is selected from the group consisting of aryl, alkylaryl, arylalkyl, cycloalkyl, heterocycloalkyl, heteroaryl, heteroarylalkyl and alkylheteroaryl, A is a chelator residue derived from a chelator selected from the group consisting of 1,4,7,10-tetraazacyclododecane-N,N′,N″,N″′-tetraacetic acid (=DOTA), N,N″-bis[2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid] (NOPO), 3,6,9,15-tetraazabicyclo[9.3.1.]pentadeca-1(15), 11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA), q is an integer of from 0-4, r is an integer from 1 to 10, preferably 2 to 5, p is an integer of from 0 to 3, preferably of from 1 to 3, Z1 is a functional moiety being attached to a carbonyl group of A and is preferably selected from the group consisting of —Y⁷—, —Y⁷—C(═Y⁶)—, —C(═Y⁶)—, —Y⁷—C(═Y⁶)—Y⁸—, —C(═Y⁶)—Y⁸—, wherein Y⁷ is selected from the group consisting of —NR^{Y7}—, —O—, —S—, —NH—NH—, —NH—O—, —CH═N—O—, —O—N═CH—, —CH═N—, —N═CH—, cyclic imides, such as -succinimide, and cyclic amines, such as

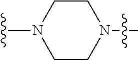

Y⁸ is selected from the group consisting of —NR^{Y8}—, —S—, —O—, —NH—NH— and Y⁶ is selected from the group consisting of NR^{Y6}, O and S, wherein R^{Y6} is H or alkyl, preferably H, and wherein R^{Y7} is H or alkyl, preferably H, and wherein R^{Y8} is H or alkyl, preferably H, and linker1 and linker2 are, independently selected from the group consisting of aryl, alkylaryl, arylalkyl, cycloalkyl, heterocycloalkyl, heteroaryl, heteroarylalkyl and alkylheteroaryl.

Further, the present invention relates to a complex comprising
(a) a radionuclide, and
(b) a PSMA binding ligand, as described above, or a pharmaceutically acceptable salt or solvate thereof.

Further, the present invention relates to a pharmaceutical composition comprising a PSMA binding ligand, as described above, or a pharmaceutically acceptable salt or solvate thereof, or the complex, as described above.

Further, the present invention relates to a PSMA binding ligand, as described above, or a pharmaceutically acceptable salt or solvate thereof, or the complex, as described above, or the pharmaceutical composition as described above, for use in treating or preventing PSMA-expressing cancers, in particular prostate cancer, and/or metastases thereof.

### The PSMA Binding Motif

As described above, the PSMA binding ligands according to the invention preferably comprise a PSMA binding motif Q.

Preferably said motif has the structure

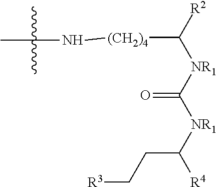

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂.

R¹ is preferably H. R², R³ and R⁴ are preferably CO₂H.

The binding motif Q thus more preferably has the structure:

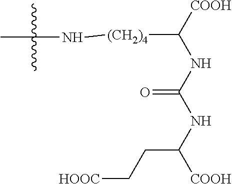

Thus, the present invention also relates to a PSMA binding ligand comprising an oligosaccharide building block which comprises a bond being cleavable by alpha-amylase, as described above, wherein this PSMA binding ligand further comprises a PSMA binding motif Q and a chelator residue A, and wherein the PSMA binding motif Q and the chelator residue A are linked via at least one linker L^{AQ} comprising the oligosaccharide building block, the PSMA binding ligand preferably having the structure

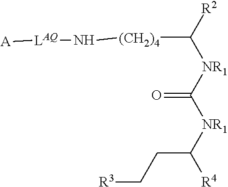

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂.

Further, the present invention relates to a PSMA binding ligand having the structure

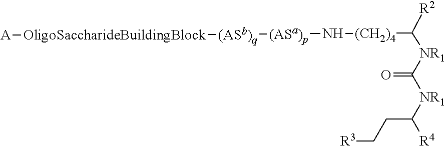

or a pharmaceutically acceptable salt or solvate thereof, wherein Q is a PSMA binding motif, A is a chelator residue, AS^{a} and AS^{b} are amino acid building blocks and q is an integer of from 0-4, preferably 0 to 3, and p is an integer of from 0 to 3, wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂.

### The Chelator Residue A

As described above, the PSMA binding ligand comprises a chelator residue A and a PSMA binding motif. The term “a chelator residue A” is denoted to mean that a chelator has been linked, via a suitable functional group to the remaining part of the PSMA binding ligand. Preferably, the chelator residue is linked to at least one linker L^{AQ} comprising the oligosaccharide building block, wherein the linker L^{AQ} in turn is linked to the PSMA binding motif.

Preferably, A is a chelator residue derived from a chelator selected from the group consisting of 1,4,7,10-tetraazacyclododecane-N,N′,N″,N″′-tetraacetic acid (=DOTA), N,N″-bis[2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid] (NOPO), 3,6,9,15-tetraazabicyclo[9.3.1.]pentadeca-1(15),11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA).

The term “chelator residue derived from a chelator selected from the group” preferably means that the above mentioned chelators, thus the chelators defined in the “group”, have been linked, via a suitable functional group functional group to the remaining part of the PSMA binding ligand. Preferably, the chelator residue is linked to at least one linker L^{AQ} comprising the oligosaccharide building block, wherein the linker L^{AQ} in turn is linked to the PSMA binding motif.

Preferably, A is a chelator residue having a structure selected from the group consisting of

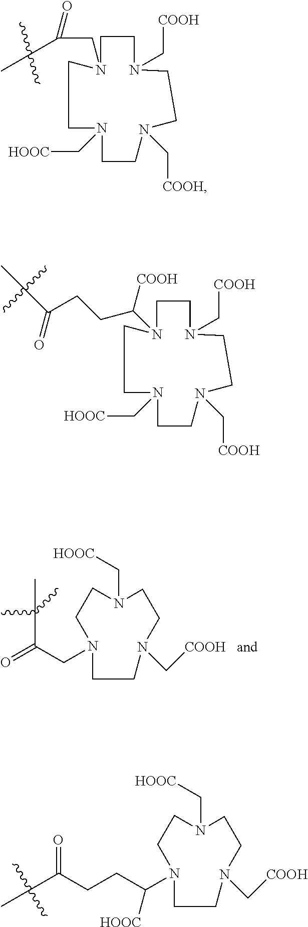 [SMILES: *C(=O)CN1CCCN(CC(=O)O)CCN(CC(=O)O)CCN(CC(=O)O)CC1 ; *C(=O)CCC(C(=O)O)N1CCCN(CC(=O)O)CCN(CC(=O)O)CCN(CC(=O)O)CC1 ; *C(=O)CN1CCN(CC(=O)O)CCN(CC(=O)O)CC1 ; *C(=O)CCC(C(=O)O)N1CCN(CC(=O)O)CCN(CC(=O)O)CC1]

Most preferably, A has the structure

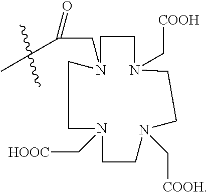 [SMILES: *C(=O)CN1CCCN(CC(=O)O)CCN(CC(=O)O)CCN(CC(=O)O)CC1]

Thus, the present invention also relates to a PSMA binding ligand comprising an oligosaccharide building block which comprises a bond being cleavable by alpha-amylase, wherein this PSMA binding ligand further comprises a PSMA binding motif Q and a chelator residue A, and wherein the PSMA binding motif Q and the chelator residue A are linked via at least one linker L^{AQ} comprising the oligosaccharide building block, the PSMA binding ligand preferably having the structure

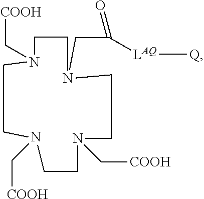

more preferably the structure

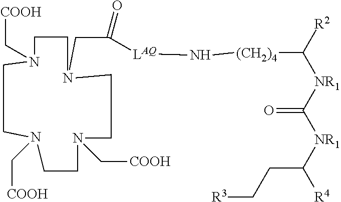

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂.

Further, the present invention relates to a PSMA binding ligand having the structure

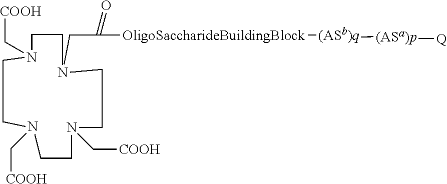

more preferably the structure

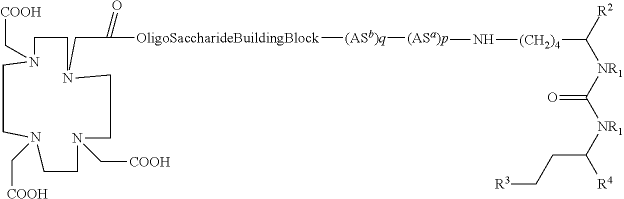

or a pharmaceutically acceptable salt or solvate thereof, wherein Q is a PSMA binding motif, A is a chelator residue, AS^{a} and AS^{b} are amino acid building blocks and q is an integer of from 0-3, and p is an integer of from 0 to 3, wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂.

### Amino Acid Building Block AS^{a}

Preferably, the PSMA binding ligand, preferably the linker L^{AQ} of the PSMA binding ligand according to structure (I), comprises an amino acid building block AS^{a}. The term “amino acid building block AS^{a}” refers to a building block consisting of at least one amino acid, preferably of from 1 to 3 amino acid, such as 1, 2 or 3 amino acids, wherein the term amino acid in this context includes any amino acid including naturally-occurring and non-naturally-occurring amino acids, such as alpha, beta and gamma amino acids, including all stereoisomeres, such as enantiomers and diastereomers of these amino acids. It is to be understood that in case, the building block AS^{a} consist of more than one amino acid, thus. e.g. of a dipeptide or tripeptide, each amino acid within the building block may be the same or may be different from each other.

Most preferably, the amino acid building block AS^{a} is an alpha amino acid. With respect to the chirality, L-amino acids are preferred.

Preferably, the amino acid building block AS^{a} has the structure

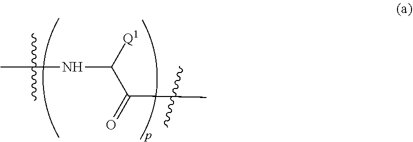

wherein p is of from 0 to 3, preferably of from 1 to 3, more preferably 1, Q¹ is selected from the group consisting of alkylaryl, arylalkyl, aryl, alkylheteroaryl, heteroarylalkyl and heteroaryl.

The term “aryl”, as used in this context of the invention, means optionally substituted, 5- and 6-membered aromatic rings, and substituted or unsubstituted polycyclic aromatic groups (aryl groups), for example tricyclic or bicyclic aryl groups. Optionally substituted phenyl groups or naphthyl groups may be mentioned as examples. Polycyclic aromatic groups can also contain non-aromatic rings.

The term “alkylaryl” as used in this context of the invention refers to aryl groups in which at least one proton has been replaced with an alkyl group (Alkyl-aryl-).

The term “arylalkyl” as used in this context of the invention refers to aryl groups linked via an alkyl group (Aryl-alkyl-).

The term “heteroaryl”, as used in this context of the invention, means optionally substituted, 5- and 6-membered aromatic rings, and substituted or unsubstituted polycyclic aromatic groups, for example tricyclic or bicyclic aryl groups, containing one or more, for example 1 to 4, such as 1, 2, 3, or 4, heteroatoms in the ring system. If more than one heteroatom is present in the ring system, the at least two heteroatoms that are present can be identical or different. Suitable heteroaryl groups are known to the skilled person. The following heteroaryl residues may be mentioned, as non limiting examples: benzodioxolyl, pyrrolyl, furanyl, thiophenyl, thiazolyl, isothiaozolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthridinyl, quinolinyl, isoqunilyinyl, indolyl, benzofuranyl, purinyl, benzofuranyl, deazapurinyl, pyridazinyl and indolizinyl.

The term “alkylheteroaryl” as used in this context of the invention refers to heteroaryl groups in which at least one proton has been replaced with an alkyl group (Alkyl-Heteroaryl-).

The term “heteroarylalkyl” as used in this context of the invention refers to heteroaryl groups linked via an alkyl group (Heteroaryl-alkyl-).

The term “cycloalkyl” means, in the context of the invention, optionally substituted, cyclic alkyl residues, wherein they can be monocyclic or polycyclic groups. Optionally substituted cyclohexyl may be mentioned as a preferred example of a cycloalkyl residue.

The term “heterocycloalkyl”, as used in this context of the invention refers to optionally substituted, cyclic alkyl residues, which have at least one heteroatom, such as O, N or S in the ring, wherein they can be monocyclic or polycyclic groups.

The terms “substituted cycloalkyl residue” or “cycloheteroalkyl”, as used in this context of the invention refers, mean cycloalkyl residues or cycloheteroalkyl residues, in which at least one H has been replaced with a suitable substituent.

Preferably, Q1 comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably Q¹ is selected from the group consisting of:

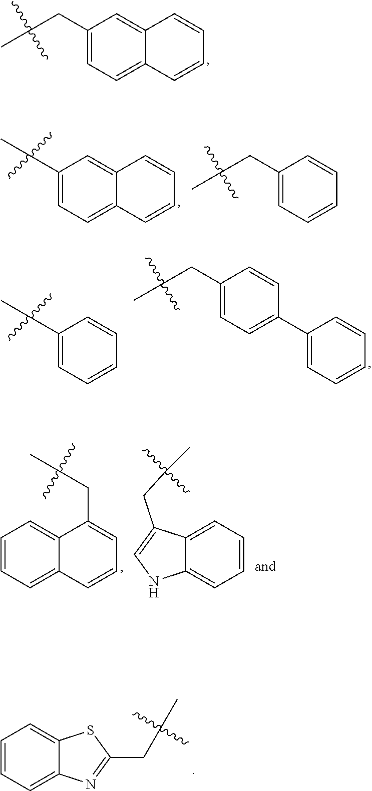

wherein Q¹ is most preferably

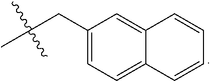 [SMILES: *Cc1ccc2ccccc2c1]

The amino acid building block AS^{a} thus preferably has the structure

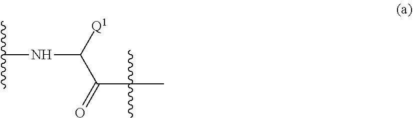

with Q¹ being

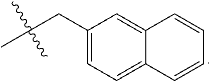 [SMILES: *Cc1ccc2ccccc2c1]

The C-terminal end of the amino acid building block AS^{a} is preferably being attached to the PSMA binding motif. In case, the PSMA binding motif has the structure

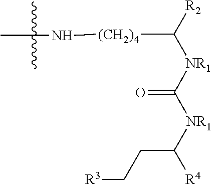

the C-terminal end of the amino acid building block AS^{a} is preferably being attached to the —NH— group of the PSMA binding motif, thereby forming the building block

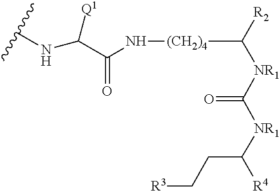

wherein R¹ is H or —CH₃, preferably H, and wherein R², R³ and R⁴ are, independently of each other, selected from the group consisting of —CO₂H, —SO₂H, —SO₃H, —OSO₃H, —PO₂H, —PO₃H and —OPO₃H₂. preferably R², R³ and R⁴ are CO₂H. It is to be understood that preferably the amino acid building block AS^{a} thus forms part of the linker linking PSMA binding motif and chelator residue mentioned above.

Thus, the present invention also relates to a PSMA binding ligand, as described above or below, comprising an oligosaccharide building block which comprises a bond being cleavable by alpha-amylase, wherein this PSMA binding ligand further comprises a PSMA binding motif Q and a chelator residue A, and wherein the PSMA binding motif Q and the chelator residue A are linked via at least one linker L^{AQ} comprising the oligosaccharide building block, wherein the linker L^{AQ} further comprises an amino acid building block AS^{a} preferably has the structure

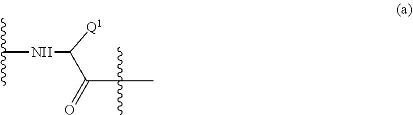

with Q¹ preferably being

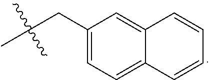 [SMILES: *Cc1ccc2ccccc2c1]

the PSMA binding ligand more preferably having the structure

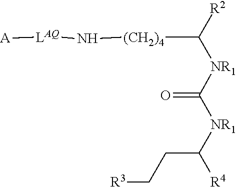

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂, and wherein the C-terminal end of the amino acid building block AS^{a} is preferably being attached to the NH group of the PSMA binding motif

Further, the present invention relates to a PSMA binding ligand, as described above or below, having the structure

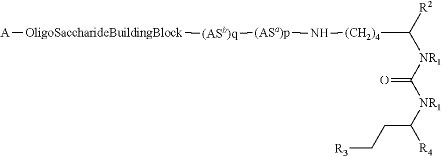

or a pharmaceutically acceptable salt or solvate thereof, wherein Q is a PSMA binding motif, A is a chelator residue, AS^{a} and AS^{b} are amino acid building blocks and q is an integer of from 0-3, and p is an integer of from 0 to 3, preferably of from 1 to 3, more preferably 1, wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂, and wherein the amino acid building block AS^{a,} has the structure

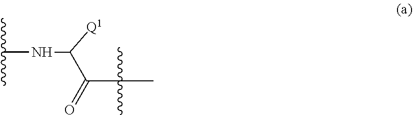

with Q¹ preferably being

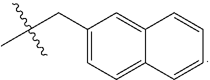 [SMILES: *Cc1ccc2ccccc2c1]

### Amino Acid Building Block AS^{b}

The PSMA binding ligand, described above and below, preferably the linker L^{AQ} of the PSMA binding ligand according to structure (I), may further comprise an amino acid building block AS^{b}. The term “amino acid building block AS^{b}” refers to a building block consisting of at least one amino acid, preferably of from 1 to 3 amino acid, such as 1, 2 or 3 amino acids, wherein the term amino acid in this context refers to any compound comprising an N-terminal (—NH—) and C-terminal end (—(C═O)—) and includes any amino acid including naturally-occurring and non-naturally-occurring amino acids, such as alpha, beta, gamma and delta amino acids, including all stereoisomeres, such as enantiomers and diastereomers of these amino acids. It is to be understood that in case, the building block AS^{a} consist of more than one amino acid, e.g. of a dipeptide or tripeptide, each amino acid within the building block may be the same or may be different from each other.

Most preferably, the amino acid building block AS^{b} has the structure (b)

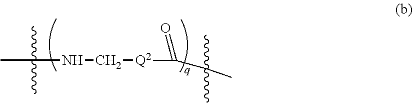

wherein Q² is selected from the group consisting of aryl, alkylaryl, arylalkyl, cycloalkyl, heterocycloalkyl, heteroaryl, heteroarylalkyl and alkylheteroaryl, and wherein q is an integer of from 0 to 4, preferably 0 to 3. It is to be understood that in case, q is >1, Q² in each building blocks may be the same or may be different from each other.

The term “aryl”, as used in this context of the invention refers to optionally substituted, 5- and 6-membered aromatic rings, and substituted or unsubstituted polycyclic aromatic groups (aryl groups), for example tricyclic or bicyclic aryl groups (—Ar—). Optionally substituted phenyl groups or naphthyl groups may be mentioned as examples. Polycyclic aromatic groups can also contain non-aromatic rings, the Aryl group in this context of the invention

The term “alkylaryl” as used in this context of the invention refers to aryl groups in which at least one proton has been replaced with an alkyl group (-alkyl-aryl-) and which are linked via to alkyl group to the —CH2-group and via the aryl group to the carbonyl group.

The term “arylalkyl” as used in this context of the invention refers to aryl groups linked via an alkyl group to the carbonyl group and via the aryl group to the —CH2-group (-aryl-alkyl-).

The term “heteroaryl” (-Heteraryl-, as used in this context of the invention, means optionally substituted, 5- and 6-membered aromatic rings, and substituted or unsubstituted polycyclic aromatic groups, for example tricyclic or bicyclic aryl groups, containing one or more, for example 1 to 4, such as 1, 2, 3, or 4, heteroatoms in the ring system. If more than one heteroatom is present in the ring system, the at least two heteroatoms that are present can be identical or different. Suitable heteroaryl groups are known to the skilled person. The following heteroaryl residues may be mentioned, as non limiting examples: benzodioxolyl, pyrrolyl, furanyl, thiophenyl, thiazolyl, isothiaozolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthridinyl, quinolinyl, isoqunilyinyl, indolyl, benzofuranyl, purinyl, benzofuranyl, deazapurinyl, pyridazinyl and indolizinyl.

The term “alkylheteroaryl” as used in this context of the invention refers to aryl groups in which at least one proton has been replaced with an alkyl group (-alkyl-heteroaryl-) and which are linked via to alkyl group to the —CH2-group and via the heteroaryl group to the carbonyl group.

The term “heteroarylalkyl” as used in this context of the invention refers to heteroaryl groups linked via an alkyl group to the carbonyl group and via the heteroaryl group to the —CH2-group (-aryl-alkyl-).

The term “cycloalkyl” (-cycloalkyl-) means, in the context of the invention, optionally substituted, cyclic alkyl residues, wherein they can be monocyclic or polycyclic groups. Optionally substituted cyclohexyl may be mentioned as a preferred example of a cycloalkyl residue.

The term “heterocycloalkyl”, as used in this context of the invention refers to optionally substituted, cyclic alkyl residues, which have at least one heteroatom, such as O, N or S in the ring, wherein they can be monocyclic or polycyclic groups.

The terms “substituted cycloalkyl residue” or “cycloheteroalkyl”, as used in this context of the invention refers, mean cycloalkyl residues or cycloheteroalkyl residues, in which at least one H has been replaced with a suitable substituent.

Preferably, Q² is an aryl group or cycloalkyl group, more preferably

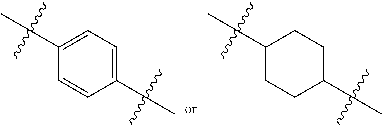 [SMILES: *c1ccc(*)cc1 ; *C1CCC(*)CC1]

most preferably

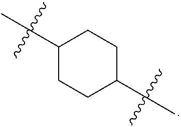 [SMILES: *C1CCC(*)CC1]

It is to be understood that any stereoisomers of Q² are possibly and included. In case Q² is

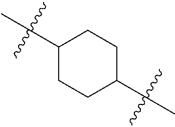 [SMILES: *C1CCC(*)CC1]

it is to be understood that this includes the cis as well as the trans isomer, with the trans isomer being particularly preferred.

Integer q is an integer of from 0-4, preferably 0-3, such 0, 1, 2 or 3, most preferably q is 0 or 1, more preferably 1.

Thus, the present invention also to a PSMA binding ligand, as described above or below, comprising an oligosaccharide building block which comprises a bond being cleavable by alpha-amylase, wherein this PSMA binding ligand further comprises a PSMA binding motif Q and a chelator residue A, and wherein the PSMA binding motif Q and the chelator residue A are linked via at least one linker L^{AQ} comprising the oligosaccharide building block, wherein the linker L^{AQ} further comprises an amino acid building block AS^{b} which preferably has the structure (b)

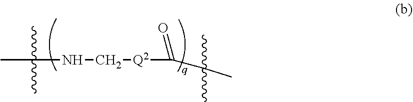

wherein Q² is selected from the group consisting of aryl, alkylaryl, arylalkyl, cycloalkyl, heterocycloalkyl, heteroaryl, heteroarylalkyl and alkylheteroaryl, and wherein q is an integer of from 0 to 4, preferably 1.

Further, the present invention relates to a PSMA binding ligand, as described above or below, having the structure

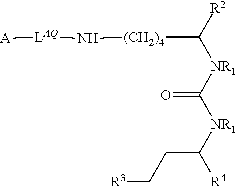

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂, wherein the linker L^{AQ} comprises the amino acid building block AS^{b,} which preferably has the structure (b)

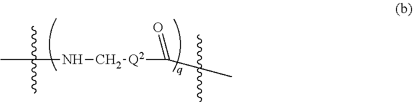

wherein Q² is selected from the group consisting of aryl, alkylaryl, arylalkyl, cycloalkyl, heterocycloalkyl, heteroaryl, heteroarylalkyl and alkylheteroaryl, and wherein q is an integer of from 0-4, preferably 1.

Further, the present invention relates to a PSMA binding ligand having the structure

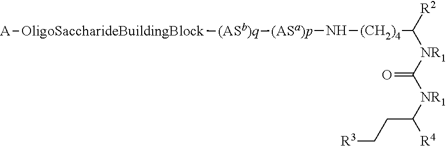

or a pharmaceutically acceptable salt or solvate thereof, wherein Q is a PSMA binding motif, A is a chelator residue, AS^{a} and AS^{b} are amino acid building blocks and q is an integer of from 0-4, preferably 1, wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂, and wherein the amino acid building block AS^{b′} has the structure (b)

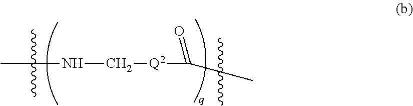

wherein Q² is selected from the group consisting of aryl, alkylaryl, arylalkyl, cycloalkyl, heterocycloalkyl, heteroaryl, heteroarylalkyl and alkylheteroaryl, and wherein q is an integer of from 0-4, preferably 1, and wherein preferably the amino acid building block AS^{a′} has the structure

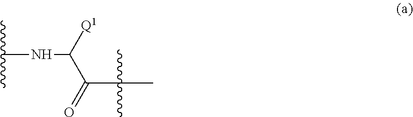

with Q¹ preferably being

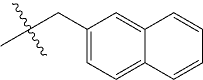 [SMILES: *Cc1ccc2ccccc2c1]

with Q² more preferably being

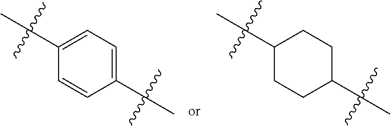 [SMILES: *c1ccc(*)cc1 ; *C1CCC(*)CC1]

more preferably

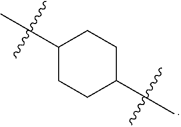 [SMILES: *C1CCC(*)CC1]

### Alpha-Amylase

Alpha-amylases, EC 3.2.1.1, are known to the skilled person as calcium metalloenzymes which break down oligosaccharides into smaller saccharide units. Alpha-amylase in mammals is found primarily in the pancreas, in salivary glands, and in saliva. Typically, alpha-amylases cleave 1,4-glycosidic linkages, in particular alpha-1,4-glucosidic linkages.

Preferably, the alpha-amylase is a secreted alpha-amylase, more preferably secreted in the saliva. Also preferably, the alpha-amylase is a mammalian alpha-amylase, more preferably a human alpha-amylase, most preferably human alpha-amylase EC 3.2.1.1. Thus, preferably, the alpha-amylase is human alpha-amylase secreted in the saliva.

### Oligosaccharide Building Block

As describe above, the PSMA binding ligand comprises an oligosaccharide building block; this oligosaccharide building block preferably comprises at least one covalent bond being cleavable by alpha-amylase (“cleavable bond”), preferably by human alpha-amylase, more preferably by human alpha-amylase EC 3.2.1.1, wherein cleavage of said cleavable bond separates the PSMA binding motif Q and the chelator residue A, i.e. removes covalent linkage between the PSMA binding motif Q and the chelator residue A. The term “cleavable by alpha-amylase”, as used herein, relates to the property of a covalent bond in the PSMA binding ligand, preferably within the oligosaccharide building block, of being hydrolysable by an alpha-amylase. Preferably, the activity of the alpha-amylase on the cleavable bond occurs at a rate corresponding to at least 0.1% , more preferably at least 1%, still more preferably at least 10%, of the activity of the alpha-amylase with maltoheptaoside in a standard assay, preferably at 25° C. in 20 mM phosphate buffer (pH 6.9) containing 6 mM NaCl (Ragunath et al. (2009), J Mol Biol 384(5):1232).

Preferably, the PSMA binding ligand comprises a PSMA binding motif Q and a chelator residue A which are linked via at least one linker L^{AQ}, wherein the at least one linker comprises the oligosaccharide building block. Thus, after cleavage of a bond present in the oligosaccharide building block by an alpha-amylase, the PSMA binding ligand is cleaved into at least two fragments, wherein one fragment comprises the PSMA binding motif Q and another fragment comprises the at least one chelator residue A. Thus, preferably, alpha-amylase activity in saliva secreted in the salivary gland causes the PSMA binding motif Q, which may potentially bind or be bound to cells of the salivary gland, to lose its covalent bondage to the chelator residue A, which is then washed away and secreted in saliva. Thus, alpha-amylase activity in the saliva removes the chelator residue A from cells of the salivary gland; in accordance, since the chelator residue A comprises the radionuclide, radio-exposition of salivary gland cells is reduced.

Thus, the oligosaccharide building block preferably comprises at least one 1,4-glycosidic linkage, more preferably a 1,4-glucosidic linkage. More preferably, the oligosaccharide building block comprises at least one alpha-1,4-glycosidic linkage, more preferably an alpha-1,4-glucosidic linkage. More preferably, the oligosaccharide building block comprises of from 2 to 10, preferably of from 3 to 6 monosaccharide units, preferably 3 monosaccharide units, wherein at least two, more preferably at least three, most preferably all, monosaccharide units are preferably linked via linkages as specified above. Thus, preferably, at least two monosaccharide units are linked with each other via 1,4-glycosidic linkages, more preferably alpha-1,4-glycosidic linkages, still more preferably alpha-1,4-glucosidic linkages. More preferably, all monosaccharide units comprised in the oligosaccharide building block are linked with their respective neighbouring monosaccharide unit or units via 1,4-glycosidic linkages, more preferably alpha-1,4-glycosidic linkages, still more preferably alpha-1,4-glucosidic linkages.

Preferably, at least two monosaccharide units present in the oligosaccharide building block form a continuous chain of monosaccharide units, preferably linked as specified herein above. Preferably, the PSMA binding motif Q and the chelator residue A are being attached, optionally via further functional groups or building blocks, to different monosaccharide units of the oligosaccharide building block, the oligosaccharide building block preferably forming a continuous chain of monosaccharide units. Preferably, the PSMA binding motif Q and the chelator residue A are being attached, optionally via further functional groups or building blocks to different monosaccharide units of the oligosaccharide building block, the oligosaccharide building block preferably forming a continuous chain of monosaccharide units, wherein the PSMA binding motif Q is attached to a terminal monosaccharide unit and the chelator residue A is attached to the other terminal monosaccharide unit of the oligosaccharide building block chain.

The term “monosaccharide”, as used herein, includes naturally-occurring and non-naturally-occurring monosaccharides capable of forming a cleavable bond as specified herein. Preferably, the monosaccharide is glucose, more preferably D-glucose, more preferably D-glucopyranose. Preferably, at least one, more preferably at least two, still more preferably at least three, even more preferably at least four, most preferably all, monosaccharide units are glucose, more preferably D-glucose, more preferably D-glucopyranose. Preferably, at least two, more preferably at least three, even more preferably at least four, most preferably all glucose units present in the oligosaccharide building block are linked with their respective neighbouring monosaccharide unit or units as specified herein above. Preferably, at least two, more preferably at least three, even more preferably at least four, most preferably all glucose units present in the oligosaccharide building block form a contiguous chain of glucose units, preferably linked as specified herein above. Preferably, the term monosaccharides includes non-natural derivatives of the aforesaid monosaccharides, wherein the PSMA binding ligand comprising said derivatives shall still have the activity of being hydrolysable by an alpha-amylase as specified herein above.

Preferably, the oligosaccharide building block comprises a maltotriose moiety. The term “maltotriose moiety”, as used herein, refers to an oligosaccharide consisting of three glucose units or derivatives of glucose units, such as substituted glucose, wherein the units are suitably linked with each other, wherein at least two glucose units are linked via an 1,4 glycosidic linkage, more preferably an alpha-1,4glucosidic linkage. More preferably, the unit comprises at least two alpha-1,4 glucosidic linkages. Preferably, the maltotriose moiety has a structure selected from structures (m1), (m2) and (m3):

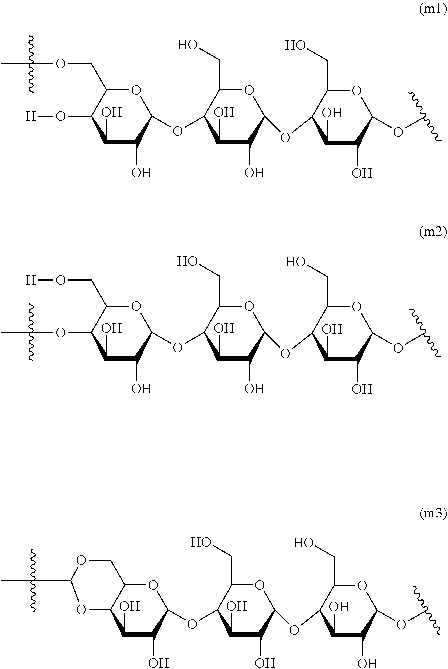

Typically, the oligosaccharide building block has the structure (c)

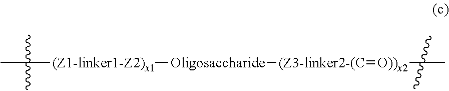

wherein Z1, Z2 and Z3 are functional groups and linker1 and linker2 are linking moieties, x1 and x2 are integers which are, independently of each other, 0, 1 or 2, preferably 0 or 1, more preferably both 1.

### The Functional Moiety Z1

The functional moiety Z1, if present, preferably forms a covalent linkage between linker1 and the chelator residue A or between linker1 and the building block (AS^{b}), preferably between linker1 and the chelator residue A.

There are in principle no restrictions as to the nature of the functional group Z1 provided that this group forms a suitable bond with the respective residue of A or AS^{b}, preferably A. Depending on the functionality of the respective chelator, the functional group Z1 is thus suitably chosen. Z1 may for example be a functional group, which, if present, is selected from the group consisting of —Y⁷—, —Y⁷—C(═Y⁶)—, —C(═Y⁶)—, —Y⁷—C(═Y⁶)—Y⁸—, —C(═Y⁶)—Y⁸—, wherein Y⁷ is selected from the group consisting of —NR^{Y7}—, —O—, —S—, —NH—NH—, —NH—O—, —CH═N—O—, —O—N═CH—, —CH═N—, —N═CH—, cyclic imides, such as -succinimide, and cyclic amines, such as

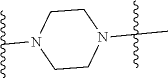 [SMILES: *N1CCN(*)CC1]

Y⁸ is selected from the group consisting of —NR^{Y8}—, —S—, —O—, —NH—NH— and Y⁶ is selected from the group consisting of NR^{Y6}, O and S, wherein R^{Y6} is H or alkyl, preferably H, and wherein R^{Y7} is H or alkyl, preferably H, and wherein R^{Y8} is H or alkyl, preferably H.

In case, Z1 is linked to chelator residue A, and in case A has a structure selected from the group consisting of

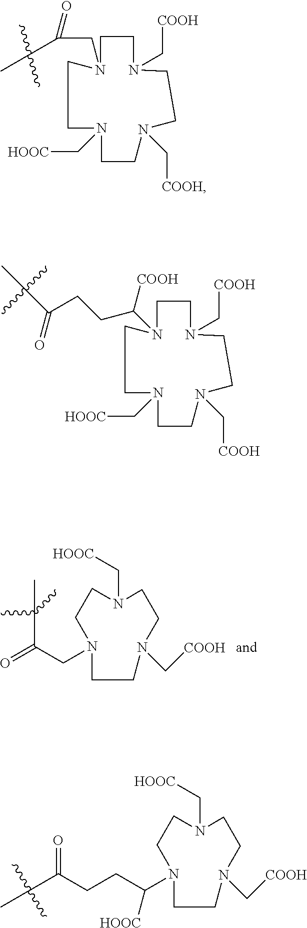 [SMILES: *C(=O)CN1CCCN(CC(=O)O)CCN(CC(=O)O)CCN(CC(=O)O)CC1 ; *C(=O)CCC(C(=O)O)N1CCCN(CC(=O)O)CCN(CC(=O)O)CCN(CC(=O)O)CC1 ; *C(=O)CN1CCN(CC(=O)O)CCN(CC(=O)O)CC1 ; *C(=O)CCC(C(=O)O)N1CCN(CC(=O)O)CCN(CC(=O)O)CC1]

Z1 is preferably a group forming a stable linkage with a carbonyl group, preferably in this case Z1 is selected from the group consisting of —NR^{Y7}—, —O—, —NH—NH—, —NH—O—, —NH—NH—C(═O)— and

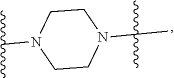 [SMILES: *N1CCN(*)CC1]

R^{Y7} is H or alkyl, preferably H.

More preferably Z1 is

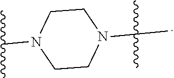 [SMILES: *N1CCN(*)CC1]

### The Functional Moiety Z2

The functional moiety Z2, if present, preferably forms a covalent linkage between linker1 and one of the terminal monosaccharides of oligosaccharide building block.

There are in principle no restrictions as to the nature of the functional group Z2 provided that this group forms a suitable bond with the respective monosaccharide. Depending on the functionality of the respective monosaccharide, the functional group Z2 is thus suitably chosen. E.g. Z2 may e.g. form with a functional group of the monosacharide a semi acetal group, an acetal group, an ether linkage, an ester linkage or a —O—CH2-CH(OH)— group. Suitable linking groups are known to the skilled person. Preferably, Z2, forms with a functional group of the monosaccharide an acetal group.

### The Linker1

Linker L¹ is a linking moiety bridging Z¹ and Z². In general, there are no particular restrictions as to the chemical nature of the linking moiety L1 with the proviso it provides suitable chemical properties for the novel conjugates for their intended use. Thus, the term “linker1” refers any suitable chemical moiety bridging Z1 and Z2 and typically includes moieties such as an alkyl, alkenyl, alkynyl, alkylaryl, arylalkyl, aryl, alkylarylalkyl, heteroaryl, alkylheteroaryl, alkylheteroalkyl, heteroarylalkyl, cycloalkyl, cycloheteroalkyl g or a peptidic group The term “alkyl” as used in the context of any linking moiety described in the present invention relates to non-branched alkyl residues, branched alkyl residues, cycloalkyl residues, as well as residues comprising one or more heteroatoms or functional groups, such as, by way of example, —O—, —S—, —NH—, —NH—C(═O)—, —C(═O)—NH—, and the like. The term also encompasses alkyl groups which are further substituted by one or more suitable substituent as well as alkoxyalkyl groups.

The term “substituted” as used within the present invention preferably refers to groups being substituted in any position by one or more substituents (replacement by a proton with the respective substituent), preferably by 1, 2, 3, 4, 5 or 6 substituents, more preferably by 1, 2, or 3 substituents. If two or more substituents are present, each substituent may be the same or may be different from the at least one other substituent. There are in general no limitations as to the substituent. The substituents may be, for example, selected from the group consisting of aryl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, amino, acylamino, including alkylcarbonylamino, arylcarbonylamino, carbamoyl and ureido, amidino, nitro, imino, sulfhydryl, alkylthio, arylthio, thiocarboxylate, sulfates, alkylsulfinyl, sulfonate, sulfamoyl, sulfonamido, trifluoromethyl, cyano, azido, cycloalkyl such as e.g. cyclopentyl or cyclohexyl, heterocycloalkyl such as e.g. morpholino, piperazinyl or piperidinyl, alkylaryl, arylalkyl and heteroaryl. Preferred substituents of such organic residues are, for example, halogens, such as fluorine, chlorine, bromine or iodine, amino groups, hydroxyl groups, carbonyl groups, thiol groups, —COOH, and —NH—(C═NH2)-NH2 groups. It is to be understood that in case, linker1 is a substituted or unsubstituted alkyl group, the structure —Z1-linker1-Z2- may e.g. be a residue derived from a natural or unnatural amino acid.

The term “aryl”, as used in the context of any linking moiety mentioned hereinunder and above refers to optionally substituted, 5- and 6-membered aromatic rings, and substituted or unsubstituted polycyclic aromatic groups (aryl groups), for example tricyclic or bicyclic aryl groups (—Ar—), which link the respective functional groups with each other. Optionally substituted phenyl groups or naphthyl groups may be mentioned as examples. Polycyclic aromatic groups can also contain non-aromatic rings, the Aryl group in this context of the invention

The term “alkenyl” as used in the context of any linking moiety described in the present invention refers to unsaturated alkyl groups having at least one double bond. The term also encompasses alkenyl groups which are substituted by one or more suitable substituent.

The term “alkynyl” as used in the context of any linking moiety described in the present invention refers to unsaturated alkyl groups having at least one triple bond. The term also encompasses alkynyl groups which are substituted by one or more suitable substituent.

The term “alkylaryl” in the context of linking moieties mentioned hereinunder and above refers to aryl groups in which at least one proton has been replaced with an alkyl group (-alkyl-aryl-) and which are linked on one side via the alkyl group and on the other side via the aryl group.

The term “arylalkyl” in the context of linking moieties mentioned hereinunder and above refers groups being linked on one side via the aryl group and on the other side via the alkyl group (-aryl-alkyl-).

The term “alkyl arylalkyl” in the context of linking moieties mentioned hereinunder and above refers groups being linked on both sides via alkyl groups, wherein the alkyl group links both alkyl residues with each other.

The term “heteroaryl” (-Heteraryl-), as used in the context of any linking moiety described in the present invention, means optionally substituted, 5- and 6-membered aromatic rings, and substituted or unsubstituted polycyclic aromatic groups, for example tricyclic or bicyclic aryl groups, containing one or more, for example 1 to 4, such as 1, 2, 3, or 4, heteroatoms in the ring system. If more than one heteroatom is present in the ring system, the at least two heteroatoms that are present can be identical or different. Suitable heteroaryl groups are known to the skilled person. The following heteroaryl residues may be mentioned, as non limiting examples: benzodioxolyl, pyrrolyl, furanyl, thiophenyl, thiazolyl, isothiaozolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthridinyl, quinolinyl, isoqunilyinyl, indolyl, benzofuranyl, purinyl, benzofuranyl, deazapurinyl, pyridazinyl and indolizinyl.

The term “alkylheteroaryl” as used in the context of any linking moiety described in the present invention refers to aryl groups in which at least one proton has been replaced with an alkyl group (-alkyl-heteroaryl-) and which are linked on one side via the alkyl group and on the other side via the heteroaryl group.

The term “heteroarylalkyl” as used in this context of the invention refers to heteroaryl groups and which are linked on one side via the heteroaryl group and on the other side via the aryl group (-heteroaryl-alkyl-).

The term “cycloalkyl” (-cycloalkyl-) means, in the context of the invention, optionally substituted, cyclic alkyl residues, wherein they can be monocyclic or polycyclic groups.

The term “heterocycloalkyl”, as used in this context of the invention refers to optionally substituted, cyclic alkyl residues, which have at least one heteroatom, such as O, N or S in the ring, wherein they can be monocyclic or polycyclic groups.

The term “peptidic group” or “peptidic structure” as used in this context of the invention refers to building blocks in which the moiety —Z1-linker1-Z2- comprises or consists of an amino acid building block, the linker1 thus comprises —NH—C(═O)— bonds in its backbone.

Preferably, the building block —Z1-linker1-Z2 in this case comprises or consists of a building block having the structure

-((X^{Z1})_{nz1}(X^{Z2})_{nz2})_{nz}-

wherein X^{Z1} and X^{Z2} are independently of each other, amino acids, preferably charged amino acids, and nz is of from 0 to 9, and nz1 and nz2 are, independently of each other, an integer of from 0 to 3.

According to a first preferred embodiment, the building block —Z1-linker¹-Z2 has the structure or consists of the structure -((X^{Z1})_{nz1}(X^{Z2})_{nz2})_{nz}-, the functional group Z1 is a functional group derived from the reaction of the N terminal end of the building block -((X^{Z1})_{nz1}(X^{Z2})_{nz2})_{nz}- with the chelator residue A or with the building block (AS^{b}), preferably with A, and the functional group Z2 is a functional group derived from the C-Terminal end of the peptidic building block and formed upon reaction of the building block with the oligosaccharide, preferably a —(C═O)— group or group forming an acetal group with two OH groups of the Saccharide, preferably the adjacent monosaccharide.

According to a second preferred embodiment, linker1 may comprise the building block -((X^{Z1})_{nz1}(X^{Z2})_{nz2})_{nz}- wherein this building block may be attached via a linker^{Z1} to Z1 and/or a linker^{Z2} to Z2, thus Z1-linker1-Z2 may have the structure Z1-(linker^{Z1})ₐ-((X^{Z1})_{nz1}(X^{Z2})_{nz2})_{nz-(linker}^{Z2})ₐₐ-Z2 with a and aa being, independently of each other, 0 or 1, and with linker^{Z1} being preferably selected from the group consisting of alkyl-(C═O)—, alkenyl-(C═O)—, alkynyl-(C═O)—, alkylaryl-(C═O)—, arylalkyl-(C═O)—, aryl-(C═O)—, alkylarylalkyl-(C═O)—, heteroaryl-(C═O)—, alkylheteroaryl-(C═O)—, alkylheteroalkyl-(C═O)—, heteroarylalkyl-(C═O)—, cycloalkyl-(C═O)— and cycloheteroalkyl-(C═O)— and with linker^{Z2} being preferably selected from the group consisting of —NH-alkyl, —NH-alkenyl, —NH-alkynyl, —NH-alkylaryl, —NH-arylalkyl, —NH-aryl, —NH-alkylarylalkyl, —NH-heteroaryl, —NH-alkylheteroaryl, —NH-alkylheteroalkyl, —NH-heteroarylalkyl, —NH-cycloalkyl and —NH-cycloheteroalkyl.

The term “charged amino acid” as used in this context of the invention refers to an amino acid that comprises a side chain that is negatively charged (i.e., de-protonated) or positively charged (i.e., protonated) in aqueous solution at physiological pH. It is to be understood that the term includes naturally-occurring and non-naturally-occurring charged amino acids, including all stereoisomeres, such as enantiomers and diastereomers of these amino acids. Most preferably, the amino acids are alpha amino acids. With respect to the chirality, L-amino acids are preferred. The term “negatively charged amino acid” includes, but is not limited to, aspartic acid, glutamic acid, cysteic acid, homocysteic acid, and homoglutamic acid, homoglutamic acid, a sulfonic acid derivative of Cys, cysteic acid, homocysteic acid, aspartic acid (D) and glutamic acid (E). The term “positively charged amino acid” includes, but is not limited to, arginine, lysine, histidine homoarginine, 3- and 4-substituted arginine analogs, N(delta)-methyl-arginine (deltaMA), canavanine, substituted analogs of canavanine, α-Amino-β-guanidinopropionic acid, γ-guanidinobutyric acid, citrulline, 3-guanidinopropionic acid, 4-{[amino(imino)methyl]amino}butanoic acid, 6-{[amino(imino)methyl]amino}hexanoic acid, 2-Amino-3-guanidinopropionic acid, Arginine hydroxamate, Agmatine (CAS #: 2482-00-0), and NG-Methyl-arginine. Typically, X^{Z1} and X^{Z2}, are, independently of each other, selected from the group consisting of aspartic acid, glutamic acid, cysteic acid, homocysteic acid, homoglutamic acid, a sulfonic acid derivative Cys, cysteic acid, homocysteic acid, aspartic acid (D), glutamic acid (E), arginine, lysine, histidine homoarginine, 3- and 4-substituted arginine analogs, N(delta)-methyl-arginine (deltaMA), canavanine, substituted analogs of canavanine, α-Amino-β-guanidinopropionic acid, γ-guanidinobutyric acid, citrulline, 3-guanidinopropionic acid, 4-{[amino(imino)methyl]amino}butanoic acid, 6-{[amino(imino)methyl]amino}hexanoic acid, 2-Amino-3-guanidinopropionic acid, Arginine hydroxamate, Agmatine (CAS #: 2482-00-0), and NG-Methyl-arginine. Preferably, X^{Z1} and X^{Z2}, are independently of each other, selected from the group consisting of aspartic acid, glutamic acid, lysine (K), histidine (H) and arginine (R). More preferably, at least one of X^{Z1} and X^{Z2} is a negatively charged, and at least one of X^{Z1} and X^{Z2} is a positively charged amino acid. Even more preferably, at least one of X^{Z1} and X^{Z2} is histidine (H) and at least one of X^{Z1} and X^{Z2} is glutamic acid (E). Even more preferably, the building block -((X^{Z1})_{nz1}(X^{Z2})_{nz2})_{nz}-, has the structure (HE)_{nz} or (EH)_{nz}, preferably (EH)_{nz}, most preferably (EH)₃.

Preferably, linker1 is an aryl group, more preferably a phenyl group, more preferably an phenyl group linked via 1,4 position to the functional groups Z1 and Z2, wherein Z2 is more preferably

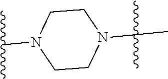 [SMILES: *N1CCN(*)CC1]

and wherein Z2 preferably forms an acetal group with two OH groups of a terminal monosaccharide moiety of the oligosaccharide.

According to a further preferred embodiment, linker1 comprises a substituted alkyl group or forms together with Z1 and Z2 a peptidic structure, as described above. In case, linker1 comprises a substituted alkyl group, the unit Z1-linker-Z2 is preferably a residue derived from an amino acid. Z1 in this case is preferably —NH— and Z2 is preferably —(C═O)— or forms an acetal group with two OH groups of the Saccharide, preferably the adjacent monosaccharide. Preferably, the unit Z1-linker-Z2, in tis case, is a residue derived from a charged amino acids. The term “charged amino acid” as used herein refers to an amino acid that comprises a side chain that is negatively charged (i.e., de-protonated) or positively charged (i.e., protonated) in aqueous solution at physiological pH. It is to be understood that the term includes naturally-occurring and non-naturally-occurring charged amino acids, including all stereoisomeres, such as enantiomers and diastereomers of these amino acids. Most preferably, the amino acids are alpha amino acids. With respect to the chirality, L-amino acids are preferred. The term “negatively charged amino acid” includes, but is not limited to, aspartic acid, glutamic acid, cysteic acid, homocysteic acid, and homoglutamic acid, homoglutamic acid, a sulfonic acid derivative of Cys, cysteic acid, homocysteic acid, aspartic acid (D) and glutamic acid (E). More preferably, the negatively charged amino acid is aspartic acid or glutamic acid (E). The term “positively charged amino acid” includes, but is not limited to, arginine, lysine, histidine homoarginine, 3- and 4-substituted arginine analogs, N(delta)-methyl-arginine (deltaMA), canavanine, substituted analogs of canavanine, α-Amino-β-guanidinopropionic acid, γ-guanidinobutyric acid, citrulline, 3-guanidinopropionic acid, 4-{[amino(imino)methyl]amino}butanoic acid, 6-{[amino(imino)methyl]amino}hexanoic acid, 2-Amino-3-guanidinopropionic acid, Arginine hydroxamate, Agmatine (CAS #: 2482-00-0), and NG-Methyl-arginine. Most preferred positively charged amino acids are lysine (K), histidine (H) or arginine (R).

### The Functional Moiety Z3

The functional moiety Z3, if present, preferably forms a covalent linkage between linker2 and one of the terminal monosaccharides of oligosaccharide building block.

There are in principle no restrictions as to the nature of the functional group Z3 provided that this group forms a suitable bond with the respective monosaccharide. Depending on the functionality of the respective monosaccharide, the functional group Z3 is thus suitably chosen. E.g. Z2 may form with a functional group of the monosacharide a semi actal group, an acetal group, an ether linkage, an ester linkage or a —O—CH2—CH(OH)— group.

### The Linker2

Linker L² is a linking moiety bridging Z³ and the carbonyl group, wherein the carbonyl group is preferably being attached to the N-terminus of AS^{b} or AS^{a}, preferably AS^{b}. In general, there are no particular restrictions as to the chemical nature of the linking moiety L2 with the proviso it provides suitable chemical properties for the novel conjugates for their intended use. Thus, the term “linker2” refers any suitable chemical moiety bridging Z³ and the carbonyl group and typically includes moieties such as an alkyl, alkenyl, alkynyl, alkylaryl, arylalkyl, aryl, alkylarylalkyl, heteroaryl, alkylheteroaryl, alkylheteroalkyl, heteroarylalkyl, cycloalkyl, cycloheteroalkyl or peptidic group.

Preferably, linker2 is an arylalkyl group, more preferably a phenylethyl group, wherein the n phenyl group is preferably linked via 1,4 position to the functional groups Z3 and the ethyl group, wherein Z3 preferably forms an ether linkage with the oligosaccharide.

According to a further preferred embodiment, linker2 comprises or consists of a substituted alkyl group or a peptidic group or structure. The term “peptidic structures” as used in this context of the invention refers to building blocks in which the moiety —Z3-linker1-C(═O)— comprises or consists of an amino acid building block, the linker1 thus comprises —NH—C(═O)— bonds in its backbone. Preferably, the building block —Z3-linker1-C(═O)— in this case comprises or consists of a building block having the structure -((X^{Z3})_{nz3}(X^{Z4})_{nz4})_{nzz}- wherein X^{Z3} and X^{Z4} are independently of each other, amino acids, preferably charged amino acids, and nzz is of from 0 to 9, and nz3 and nz4, are independently of each other, an integer of from 0 to 3.

According to a first preferred embodiment, the building block —Z3-linker1-C(═O)— has or consists of the structure -((X^{Z3})_{nz3}(X^{Z4})_{nz4})_{nzz}-. In this case, the functional group Z3 is a functional group derived from the reaction of the N terminal end of the building block -((X^{Z3})_{nz3}(X^{Z4})_{nz4})_{nzz}- with the oligosaccharide.

According to a second preferred embodiment, linker2 comprises the building block -((X^{Z3})_{nz3}(X^{Z4})_{nz4})_{nzz}- wherein this building block may be attached via a linker^{Z3} to Z3 and/or a linker^{Z4} to the carbonyl group, thus Z3-linker1-C(═O)— may have the structure Z3-(linker^{Z3})_{b}-((X^{Z3})_{nz3}(X^{Z4})_{nz4})_{nzz}-(linker^{Z4})_{bb}-C(═O)— with b and bb being, independently of each other 0 or 1, and with linker^{Z3} being a linking moiety, preferably selected from the group consisting of alkyl-(C═O)—, alkenyl-(C═O)—, alkynyl-(C═O)—, alkylaryl-(C═O)—, arylalkyl-(C═O)—, aryl-(C═O)—, alkylarylalkyl-(C═O)—, heteroaryl-(C═O)—, alkylheteroaryl-(C═O)—, alkylheteroalkyl-(C═O)—, heteroarylalkyl-(C═O)—, cycloalkyl-(C═O)— and cycloheteroalkyl-(C═O)— and with linker^{Z4} being a linking moiety, preferably selected from the group consisting of NH-alkyl, —NH-alkenyl, —NH-alkynyl, —NH-alkylaryl, —NH-arylalkyl, —NH-aryl, —NH-alkylarylalkyl, —NH-heteroaryl, —NH-alkylheteroaryl, —NH-alkylheteroalkyl, —NH-heteroarylalkyl, —NH-cycloalkyl and —NH-cycloheteroalkyl.

Preferably bb in this case is 0. The term “charged amino acid” as used in this context of the invention refers to an amino acid that comprises a side chain that is negatively charged (i.e., de-protonated) or positively charged (i.e., protonated) in aqueous solution at physiological pH. It is to be understood that the term includes naturally-occurring and non-naturally-occurring charged amino acids, including all stereoisomeres, such as enantiomers and diastereomers of these amino acids. Most preferably, the amino acids are alpha amino acids. With respect to the chirality, L-amino acids are preferred. The term “negatively charged amino acid” includes, but is not limited to, aspartic acid, glutamic acid, cysteic acid, homocysteic acid, and homoglutamic acid, homoglutamic acid, a sulfonic acid derivative of Cys, cysteic acid, homocysteic acid, aspartic acid (D) and glutamic acid (E). The term “positively charged amino acid” includes, but is not limited to, arginine, lysine, histidine homoarginine, 3- and 4-substituted arginine analogs, N(delta)-methyl-arginine (deltaMA), canavanine, substituted analogs of canavanine, α-Amino-β-guanidinopropionic acid, γ-guanidinobutyric acid, citrulline, 3-guanidinopropionic acid, 4-{[amino(imino)methyl]amino}butanoic acid, 6-{[amino(imino)methyl]amino}hexanoic acid, 2-Amino-3-guanidinopropionic acid, Arginine hydroxamate, Agmatine (CAS #: 2482-00-0), and NG-Methyl-arginine. Typically, X^{Z3} and X^{Z4}, are, independently of each other, selected from the group consisting of aspartic acid, glutamic acid, cysteic acid, homocysteic acid, homoglutamic acid, a sulfonic acid derivative Cys, cysteic acid, homocysteic acid, aspartic acid (D), glutamic acid (E), arginine, lysine, histidine homoarginine, 3- and 4-substituted arginine analogs, N(delta)-methyl-arginine (deltaMA), canavanine, substituted analogs of canavanine, α-Amino-β-guanidinopropionic acid, γ-guanidinobutyric acid, citrulline, 3-guanidinopropionic acid, 4-{[amino(imino)methyl]amino}butanoic acid, 6-{[amino(imino)methyl]amino}hexanoic acid, 2-Amino-3-guanidinopropionic acid, Arginine hydroxamate, Agmatine (CAS #: 2482-00-0), and NG-Methyl-arginine. Preferably, X^{Z3} and X^{Z4}, are independently of each other, selected from the group consisting of aspartic acid, glutamic acid, lysine (K), histidine (H) and arginine (R). More preferably, at least one of X^{Z3} and X^{Z4} is a negatively charged, and at least one of X^{Z3} and X^{Z4} is a positively charged amino acid. Even more preferably, at least one of X^{Z3} and X^{Z4} is histidine (H) and at least one of X^{Z3} and X^{Z4} is glutamic acid (E). Even more preferably, the building block -((X^{Z3})_{nz3}(X^{Z4})_{nz4})_{nzz}-, has the structure (HE)_{nz} or (EH)_{nz}, preferably (EH)_{nz}, most preferably (EH)3.

In case, linker2 comprises or consists of a substituted alkyl group the unit Z3-linker-C(═O)— is preferably a residue derived from an amino acid. Z3 in this case is preferably a residue derived from a charged amino acid. The term “charged amino acid” as used in this context refers to an amino acid that comprises a side chain that is negatively charged (i.e., de-protonated) or positively charged (i.e., protonated) in aqueous solution at physiological pH. It is to be understood that the term includes naturally-occurring and non-naturally-occurring charged amino acids, including all stereoisomeres, such as enantiomers and diastereomers of these amino acids. Most preferably, the amino acids are alpha amino acids. With respect to the chirality, L-amino acids are preferred. The term “negatively charged amino acid” includes, but is not limited to, aspartic acid, glutamic acid, cysteic acid, homocysteic acid, and homoglutamic acid, homoglutamic acid, a sulfonic acid derivative of Cys, cysteic acid, homocysteic acid, aspartic acid (D) and glutamic acid (E). More preferably, the negatively charged amino acid is aspartic acid or glutamic acid (E). The term “positively charged amino acid” includes, but is not limited to, arginine, lysine, histidine homoarginine, 3- and 4-substituted arginine analogs, N(delta)-methyl-arginine (deltaMA), canavanine, substituted analogs of canavanine, α-Amino-β-guanidinopropionic acid, γ-guanidinobutyric acid, citrulline, 3-guanidinopropionic acid, 4-{[amino(imino)methyl]amino}butanoic acid, 6-{[amino(imino)methyl]amino}hexanoic acid, 2-Amino-3-guanidinopropionic acid, Arginine hydroxamate, Agmatine (CAS #: 2482-00-0), and NG-Methyl-arginine. Most preferred positively charged amino acids are lysine (K), histidine (H) or arginine (R).

According to a preferred embodiment of the invention, the PSMA binding ligand, as described above or below, comprises an oligosaccharide building block having the structure:

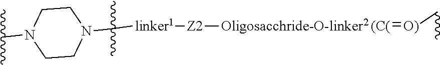

with linker1, linker2 and Z2 being as described above.

More preferably, the PSMA binding ligand, as described above or below, comprises an oligosaccharide building block having the structure

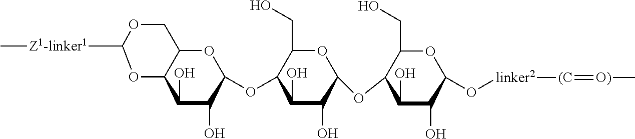

More preferably, the PSMA binding ligand, as descried above or below, comprises an oligosaccharide building block having the structure

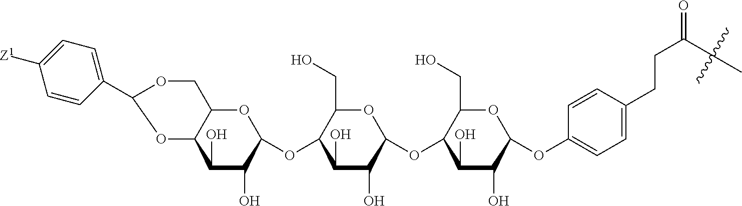

More preferably, the PSMA binding ligand, as described above or below, comprises an oligosaccharide building block having the structure

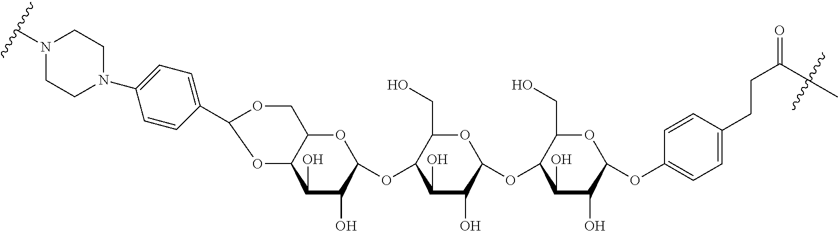

### Further Building Blocks

The PSMA binding ligand, in particular the linker L^{AQ}, may comprise further building blocks, such as amino acid building blocks which may e.g. act as pK modifier.

### Amino Acid Building Block

As amino acid building block, e.g., a building block having the structure ((X¹)ₙ₁(X²)ₙ₂)ₙ is preferred, wherein X¹ and X² are independently of each other, amino acids, preferably charged amino acids, and n is of from 0 to 9, and n1 and n2, are independently of each other, an integer of from 0 to 3. This building block may typically be present between A and the oligosaccharide building block and/or between the oligosaccharide building block and (ASb) and/or between (AS^{b}) and (AS^{a}).

Preferably, X1 and X2, are, independently of each other, selected from the group consisting of aspartic acid, glutamic acid, cysteic acid, homocysteic acid, homoglutamic acid, a sulfonic acid derivative Cys, cysteic acid, homocysteic acid, aspartic acid (D), glutamic acid (E), arginine, lysine, histidine homoarginine, 3- and 4-substituted arginine analogs, N(delta)-methyl-arginine (deltaMA), canavanine, substituted analogs of canavanine, α-Amino-β-guanidinopropionic acid, γ-guanidinobutyric acid, citrulline, 3-guanidinopropionic acid, 4-{[amino(imino)methyl]amino}butanoic acid, 6-{[amino(imino)methyl]amino}hexanoic acid, 2-Amino-3-guanidinopropionic acid, Arginine hydroxamate, Agmatine (CAS #: 2482-00-0), and NG-Methyl-arginine.

Preferably, X1 and X2, are independently of each other, selected from the group consisting of aspartic acid, glutamic acid, lysine (K), histidine (H) and arginine (R).

Preferably, at least one of X¹ or X² according to embodiment (1a) is a negatively charged, and at least one of X¹ and X² is a positively charged amino acid.

More preferably, at least one of X¹ and X² is histidine (H) and at least one of X¹ and X² is glutamic acid (E). Even more preferably, the building block ((X¹)ₙ₁(X²)ₙ₂)ₙ, has the structure (HE)ₙ or (EH)ₙ, preferably (EH)ₙ.

Thus, the present invention relates to a PSMA binding ligand comprising an oligosaccharide building block which comprises a bond being cleavable by alpha-amylase as well as a building block having the structure ((X¹)ₙ₁(X²))ₙ₂)ₙ, wherein least one of X¹ and X² is histidine (H) and at least one of X¹ and X² is glutamic acid (E) and wherein n is of from 0 to 9, and n1 and n2, are independently of each other, an integer of from 0 to 3.

Typically, this PSMA binding ligand further comprises a PSMA binding motif Q and a chelator residue A, wherein the PSMA binding motif Q and the chelator residue A are preferably linked via at least one linker L^{AQ} comprising the oligosaccharide building block, the PSMA binding ligand thus preferably having the structure (I)

A-L^{AQ}-Q

And wherein L^{AQ} further comprises a building block having the structure ((X¹)ₙ₁(X²)ₙ₂)ₙ₂, wherein least one of X¹ and X² is histidine (H) and at least one of X¹ and X² is glutamic acid (E) and wherein n is of from 0 to 9, and n1 and n2, are independently of each other, an integer of from 0 to 3. Preferably, the building ((X¹)ₙ₁(X²)ₙ₂)ₙ is (HE)₃ or (EH)₃, more preferably (EH)₃.

As described above, according to a particularly preferred embodiment, the present invention relates to a PSMA binding ligand of formula (II)

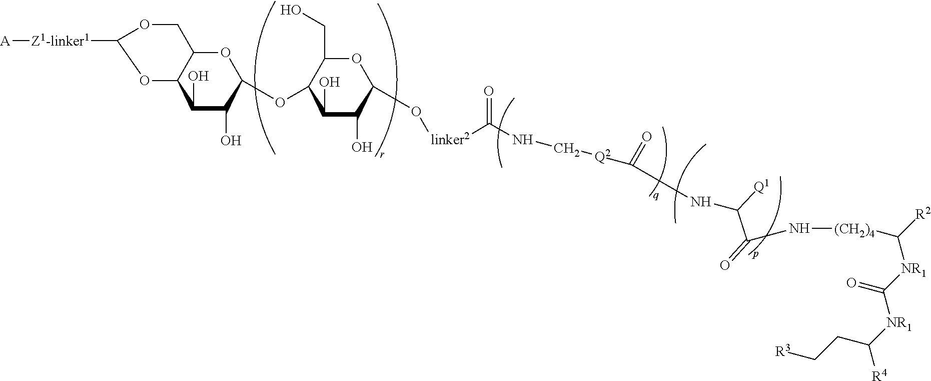

or a pharmaceutically acceptable salt or solvate thereof, wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂, Q¹ is selected from the group consisting of alkylaryl, arylalkyl, aryl, alkylheteroaryl, heteroarylalkyl and heteroaryl, Q² is selected from the group consisting of aryl, alkylaryl, arylalkyl, cycloalkyl, heterocycloalkyl, heteroaryl, heteroarylalkyl and alkylheteroaryl, A is a chelator residue derived from a chelator selected from the group consisting of 1,4,7,10-tetraazacyclododecane-N,N′,N″,N′″-tetraacetic acid (=DOTA), N,N″-bis[2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid] (NOPO), 3,6,9,15-tetraazabicyclo[9.3.1.]pentadeca-1(15),11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA), q is an integer of from 0-4, preferably 1, r is an integer from 1 to 10, preferably 2 to 5, p is an integer of from 0 to 3, preferably of from 1 to 3, Z1 is a functional moiety being attached to a carbonyl group of A and is preferably selected from the group consisting of Y⁷—, —Y⁷—C(═Y⁶)—, —C(═Y⁶)—, —Y⁷—C(═Y⁶)—Y⁸-, —C(═Y⁶)—Y⁸-, wherein Y⁷ is selected from the group consisting of —NR^{Y7}—, —O—, —S—, —NH—NH—, —NH—O—, —CH═N—O—, —O—N═CH—, —CH═N—, —N═CH—, cyclic imides, such as—succinimide, and cyclic amines, such as

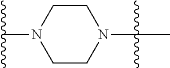 [SMILES: *N1CCN(*)CC1]

Y⁸ is selected from the group consisting of —NR^{Y8}—, —S—, —O—, —NH—NH— and Y⁶ is selected from the group consisting of NR^{Y6}, O and S, wherein R^{Y6} is H or alkyl, preferably H, and wherein R^{Y7} is H or alkyl, preferably H, and wherein R⁸ is H or alkyl, preferably H,
and linker1 and linker2 are, independently selected from the group consisting of aryl, alkylaryl, arylalkyl, cycloalkyl, heterocycloalkyl, heteroaryl, heteroarylalkyl and alkylheteroaryl.

Preferably, A is a chelator residue having a structure selected from the group consisting of

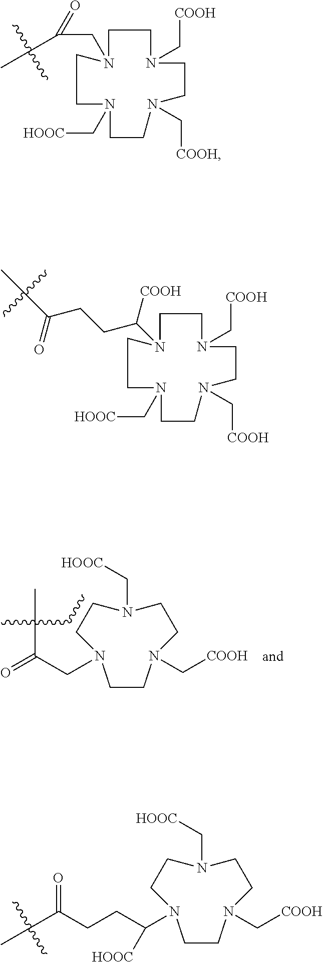

More preferably A is

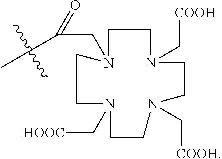 [SMILES: *C(=O)CN1CCN(CC(=O)O)CCN(CC(=O)O)CCN(CC(=O)O)CC1]

The PSMA binding ligand, thus preferably having the structure:

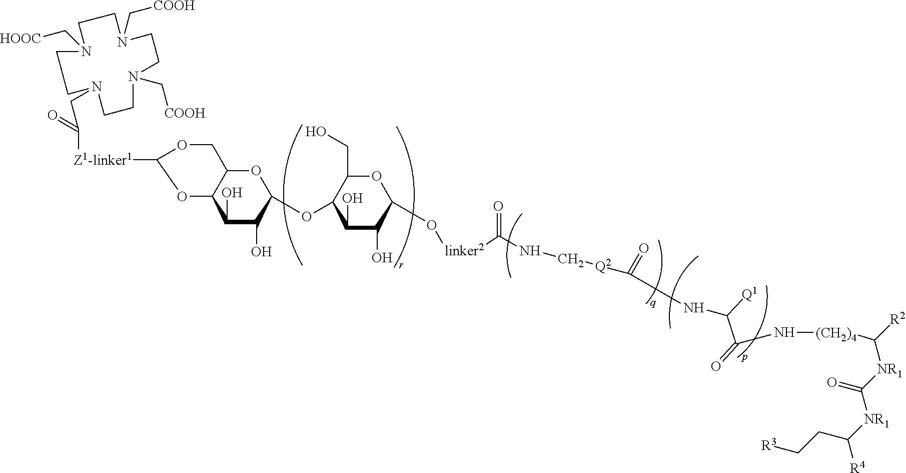

As described above, Q¹ preferably comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably wherein Q¹ is selected from the group consisting of

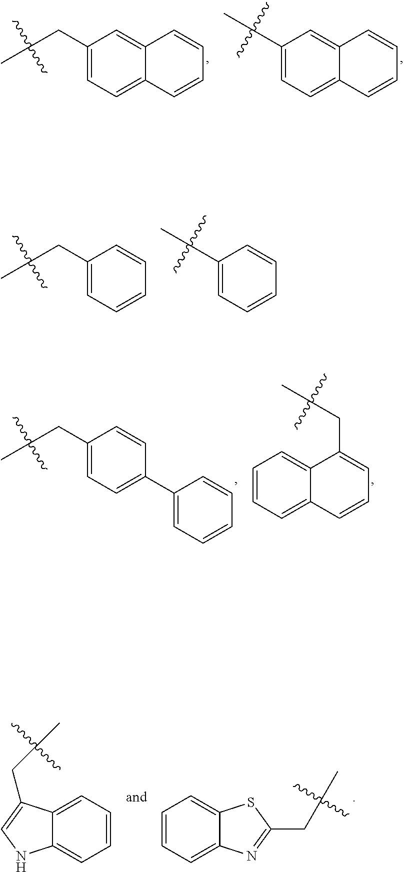

More preferably wherein Q¹ is

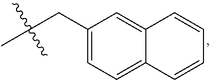 [SMILES: *Cc1ccc2ccccc2c1]

### and Q² is

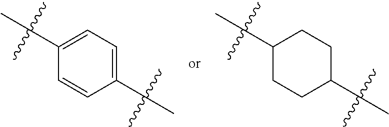 [SMILES: *c1ccc(*)cc1 ; *C1CCC(*)CC1]

preferably

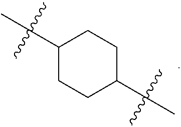 [SMILES: *C1CCC(*)CC1]

Most preferably integers q and p are both 1, and r is 2, the PSMA binding ligand, thus preferably having the structure:

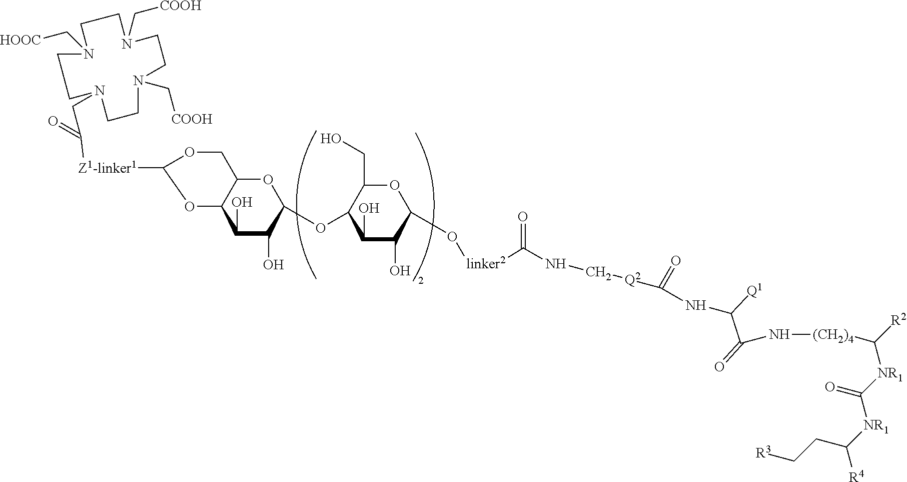

with Q¹ being

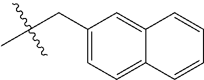 [SMILES: *Cc1ccc2ccccc2c1]

and Q2 being

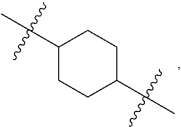 [SMILES: *C1CCC(*)CC1]

preferably in trans conformation, and wherein Z1 is preferably

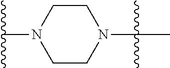 [SMILES: *N1CCN(*)CC1]

and linker1 and aryl group, and linker2 is preferably an arylalkyl group.

Most preferably the compound has the structure:

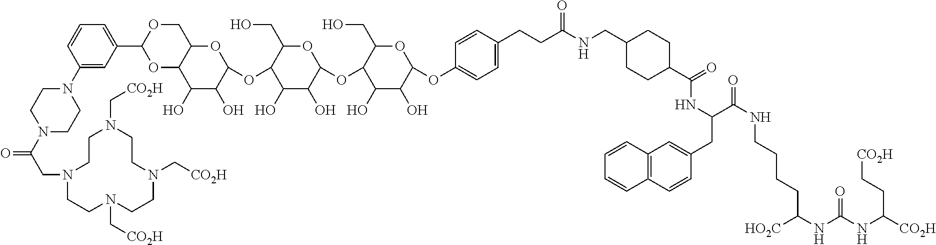 [SMILES: O=C(O)CCC(NC(=O)NC(CCCCNC(=O)C(Cc1ccc2ccccc2c1)NC(=O)C1CCC(CNC(=O)CCc2ccc(OC3OC(CO)C(OC4OC(CO)C(OC5OC6COC(c7cccc(N8CCN(C(=O)CN9CCN(CC(=O)O)CCN(CC(=O)O)CCN(CC(=O)O)CC9)CC8)c7)OC6C(O)C5O)C(O)C4O)C(O)C3O)cc2)CC1)C(=O)O)C(=O)O]

### Complex

As described above, the present invention also relates to a complex comprising
(a) a radionuclide, and
(b) a PSMA binding ligand, as described above or below, or a pharmaceutically acceptable salt or solvate thereof.

Typical pharmaceutically acceptable salts include those salts prepared by reaction of the compounds of the present invention with a pharmaceutically acceptable mineral or organic acid or an organic or inorganic base. Such salts are known as acid addition and base addition salts. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, napththalene-2-sulfonate, mandelate and the like. Preferred pharmaceutically acceptable acid addition salts are those formed with mineral acids such as hydrochloric acid and hydrobromic acid, and those formed with organic acids such as maleic acid and methanesulfonic acid. Salts of amine groups may also comprise quaternary ammonium salts in which the amino nitrogen carries a suitable organic group such as an alkyl, alkenyl, alkynyl, or aralkyl moiety. Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred. It should be recognized that the particular counter ion forming a part of any salt of this invention is usually not of a critical nature, so long as the salt as a whole is pharmacologically acceptable and as long as the counter ion does not contribute undesired qualities to the salt as a whole.

The term “pharmaceutically acceptable solvate” encompasses also suitable solvates of the compounds of the invention, wherein the compound combines with a solvent such as water, methanol, ethanol, DMSO, acetonitrile or a mixture thereof to form a suitable solvate such as the corresponding hydrate, methanolate, ethanolate, DMSO solvate or acetonitrilate.

### The Radionuclide

Depending on whether the PSMA binding ligands of the invention are to be used as radio-imaging agents or radio-pharmaceuticals different radionuclides are complexed to the chelator.

The complexes of invention may contain one or more radionuclides, preferably one radionuclide. These radionuclides are preferably suitable for use as radio-imaging agents or as therapeutics for the treatment of proliferating cells, for example, PSMA expressing cancer cells, in particular PSMA-expressing prostate cancer cells. According to the present invention they are called “metal complexes” or “radiopharmaceuticals”.

Preferred imaging methods are positron emission tomography (PET) or single photon emission computed tomography (SPECT).

Preferably, the at least one radionuclide is selected from the group consisting ⁸⁹Zr, 44Sc, ¹¹¹ln, ⁹⁰Y, ⁶⁶Ga, ⁶⁷Ga ⁶⁸Ga, ¹⁷⁷Lu, ^{99m}Tc, ⁶⁰Cu, ⁶¹Cu, ⁶²Cu, ⁶⁴Cu, ⁶⁶Cu, ⁶⁷Cu, ¹⁴⁹Tb, ¹⁵²Tb, ¹⁵⁵Tb, ¹⁵³Sm, ¹⁶¹Tb, ¹⁵³Gd, ¹⁵⁵Gd, ¹⁵⁷_{Gd,} ²¹³Bi, ²²⁵Ac, ²³⁰U, ²²³Ra, ¹⁶⁵Er, ⁵²Fe, ⁵⁹Fe and radionuclides of Pb (such as ²⁰³Pb and ²¹²Pb, ²¹¹Pb, ²¹³Pb, ²¹⁴Pb, ²⁰⁹Pb, ¹⁹⁸Pb, ¹⁹⁷Pb).

More preferably, the at least one radionuclide is selected from the group consisting ⁹⁰Y, ⁶⁸Ga, ¹⁷⁷Lu, ²²⁵Ac, and ²¹³Bi. More preferably, the radionuclide is ¹⁷⁷Lu or ²²⁵Ac.

Preferably, the radionuclide has a half-life of at least 30 min, more preferably of at least 1 h, more preferably at least 12 h, even more preferably at least 1 d, most preferably at least 5 d; also preferably, the radionuclide has a half-life of at most 1 year, more preferably at most 6 months, still more preferably at most 1 month, even more preferably at most 14 d. Thus, preferably, the radionuclide has a half-life of from 30 min to 1 year, more preferably of 12 h to 6 months, even more preferably of from 1 d to 1 month, most preferably of from 5 d to 14 d.

Preferably, the radionuclide is an α- and/or β-emitter, i.e. the radionuclide preferably emits α-particles (α-emitter) and/or β-radiation β-emitter).

Preferably, in case the radionuclide is an α-emitter, the α-particle has an energy of from 1 to 10 MeV, more preferably of from 2 to 8 MeV, most preferably of from 4 to 7 MeV.

Preferably, in case the radionuclide is a β-emitter, the β-radiation has an energy of from 0.1 to 10 MeV, more preferably of from 0.25 to 5 MeV, most preferably of from 0.4 to 2 MeV.

Preferred radionuclides emitting β-radiation are selected from the group consisting of ⁹⁰Y, ¹⁷⁷Lu, ⁵⁹Fe, 66Cu, ⁶⁷Cu, ¹⁶¹Tb, ¹⁵³Sm, ²¹²Pb, ²¹¹Pb, ²¹³Pb, ²¹⁴Pb, ²⁰⁹Pb, Very preferred radionuclides emitting β-radiation are ¹⁷⁷Lu or ⁹⁰Y, most preferably ¹⁷⁷Lu. Preferably in this case the use is diagnosis or therapy.

Preferred radionuclides emitting α-radiation are e.g. selected from the group consisting of ²¹³Bi, ²²⁵Ac, ¹⁴⁹Tb, ²³⁰U and ²²³Ra. ²¹³Bi, ²³⁰U, more preferably the radionuclide is ²²⁵Ac and/or ²¹³Bi. A very preferred radionuclide emitting α-radiation is e.g. ²²⁵AC. Preferably in this case the use is therapy.

According to a further embodiment, the radionuclide is a positron emitter. In this case the radionuclide is preferably selected from the group consisting ⁸⁹Zr, ⁴⁴Sc, ⁶⁶Ga, ⁶⁸Ga and ⁶⁴Cu. In this case, the use is preferably PET diagnosis.

According to a further preferred embodiment, radionuclide is a gamma emitter. In this case the radionuclide is preferably selected from the group consisting ¹¹¹In, ⁶⁷Ga, ^{99m}Tc, ¹⁵⁵Tb, ¹⁶⁵Er and ²⁰³Pb. In this case, the use preferably is SPECT diagnosis.

According to a further preferred embodiment, the radionuclide emits Auger electrons, and preferably decays by electron capture. In this case, the radionuclide is preferably selected from the group consisting of ⁶⁷Ga, ¹⁵⁵Tb, ¹⁵³Gd, ¹⁶⁵Er and ²⁰³Pb. In this case, the use is preferably therapy.

### Pharmaceutical Composition

As described above, the present invention also relates to a pharmaceutical composition comprising a PSMA binding ligand as described above or below, or a complex as described above or below. It is to be understood that the pharmaceutical compositions preferably comprise therapeutically effective amounts of the PSMA binding ligand and/or the complex, respectively. The pharmaceutical composition may further comprise at least one organic or inorganic solid or liquid and/or at least one pharmaceutically acceptable carrier.

The terms “medicament” and “pharmaceutical composition”, as used herein, relate to the PSMA binding ligands and/or complexes of the present invention and optionally one or more pharmaceutically acceptable carrier, i.e. excipient. The PSMA binding ligands of the present invention can be formulated as pharmaceutically acceptable salts; salts have been described herein above. The pharmaceutical compositions are, preferably, administered locally (e.g. intra-tumorally), topically or systemically. Suitable routes of administration conventionally used for drug administration are oral, intravenous, or parenteral administration as well as inhalation. A preferred route of administration is parenteral administration. A “parenteral administration route” means modes of administration other than enteral and topical administration, usually by injection, and includes, without limitation, intravenous, intramusclular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Preferably, administration is by intravenous administration or infusion. However, depending on the nature and mode of action of a PSMA binding ligand, the pharmaceutical compositions may be administered by other routes as well.

Moreover, the PSMA binding ligands can be administered in combination with other drugs either in a common pharmaceutical composition or as separated pharmaceutical compositions wherein said separated pharmaceutical compositions may be provided in form of a kit of parts. The PSMA binding ligands are, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables.

The excipient(s) must be acceptable in the sense of being compatible with the other ingredients of the formulation and, within the scope of sound medical judgment, suitable for use in contact with the tissues of a patient without excessive toxicity, irritation, allergic response, or other problem or complication, commensurate with a reasonable benefit/risk ratio. Preferably, an excipient is being not deleterious to the recipient thereof. The excipient employed may be, for example, a solid, a gel or a liquid carrier. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa. The diluent(s) is/are selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, non-immunogenic stabilizers and the like. When solutions for infusion or injection are used, they are preferably aqueous solutions or suspensions, it being possible to produce them prior to use, e.g. from lyophilized preparations which contain the active substance as such or together with a carrier, such as mannitol, lactose, glucose, albumin and the like. The readymade solutions are sterilized and, where appropriate, mixed with excipients, e.g. with preservatives, stabilizers, emulsifiers, solubilizers, buffers and/or salts for regulating the osmotic pressure. The sterilization can be obtained by sterile filtration using filters having a small pore size according to which the composition can be lyophilized, where appropriate. Small amounts of antibiotics can also be added to ensure the maintenance of sterility.

A therapeutically effective dose refers to an amount of the PSMA binding ligands to be used in a pharmaceutical composition of the present invention which prevents, ameliorates or treats the symptoms accompanying a disease or condition referred to in this specification. Therapeutic efficacy and toxicity of such PSMA binding ligands can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The dosage regimen will be determined by the attending physician and other clinical factors; preferably in accordance with any one of the above described methods. As is well known in the medical arts, dosages for any one patient depends upon many factors, including the patient's size, body surface area, age, the particular PSMA binding ligand to be administered, sex, time and route of administration, general health, and other drugs being administered concurrently. Progress can be monitored by periodic assessment. Preferred doses are specified herein below. Progress can be monitored by periodic assessment. The pharmaceutical compositions and formulations referred to herein are administered at least once in order to treat or prevent a disease or condition recited in this specification. However, the said pharmaceutical compositions may be administered more than one time, for example from one to ten times. Preferably, the pharmaceutical compositions may be administered at a frequency of once every one to six months, more preferably once every two to four months. Specific pharmaceutical compositions are prepared in a manner well known in the pharmaceutical art and comprise at least one active PSMA binding ligand referred to herein above in admixture or otherwise associated with a pharmaceutically acceptable carrier or diluent. For making those specific pharmaceutical compositions, the active PSMA binding ligand(s) will usually be mixed with a carrier or the diluent, or enclosed or encapsulated in a capsule, sachet, cachet, paper or other suitable containers or vehicles. The resulting formulations are to be adapted to the mode of administration, i.e. in the forms of tablets, capsules, suppositories, solutions, suspensions or the like. Dosage recommendations shall be indicated in the prescribers or users instructions in order to anticipate dose adjustments depending on the considered recipient.

The term “patient”, as used herein, relates to a vertebrate, preferably a mammalian animal, more preferably a human, monkey, cow, horse, cat or dog. Preferably, the mammal is a primate, more preferably a monkey, most preferably a human).

The dosage of the PSMA binding ligand according to formula (1) administered to a patient, preferably, is defined as a compound dosage, i.e. the amount of compound administered to the patient. Preferred diagnostic compound dosages are total doses of 1-10 nmol/patient; thus, preferably, the diagnostic compound dosage is of from 0.02 to 0.1 nmol/kg body weight. Preferred therapeutic compound dosages are total doses of 10 to 100 nmol/patient; thus, preferably, the therapeutic compound dosage is of from 0.2 to 1 nmol/kg body weight.

As will be understood by the skilled person, the dosage of the complex as specified herein, i.e. a complex comprising, preferably consisting of, a radionuclide and a PSMA binding ligand according to formula (1), preferably is indicated as compound dosage as specified above, preferred dosages being the same as specified above. More preferably, the dosage of the complex is indicated as activity dosage, i.e. as the amount of radioactivity administered to the patient. Preferably, the activity dosage is adjusted such as to avoid adverse effects as specified elsewhere herein. Preferably, a patient-specific dose, preferably a patient-specific activity dosage, is determined taking into account relevant factors as specified elsewhere herein, in particular taking into account therapeutic progress and/or adverse effects observed for the respective patient. Thus, preferably, the activity dosage is adjusted such that the organ-specific dose in salivary glands is at most 30 Sv, more preferably less than 20 Sv, still more preferably less than 10 Sv, most preferably less than 5 Sv,

The effective amount may be administered once (single dosage) with an activity dosage of from about 2 MBq to about 30 MBq, preferably 4 to 30 Mbq, more preferably 6 to 30 Mbq, more preferably 8 to 30 Mbq , more preferably 10 to 30 Mbq, more preferably 15 to 30 Mbq, preferably 20 to 30 Mbq to the patient. Thus, a preferred therapeutic dose in such case is of from 2 MBq to about 30 MBq/patient, preferably 4 to 30 Mbq/patient, more preferably 6 to 30 Mbq/patient, more preferably 8 to 30 Mbq/patient, more preferably 10 to 30 Mbq/patient, more preferably 15 to 30 Mbq/patient, preferably 20 to 30 Mbq/patient. Preferably said activity dosage ranges from about 10 to 30 MBq per administration, such as for example about 10, 11, 12, 13, 14, 15, 16, 17, 18, 19, 20, 21, 22, 23, 24, 25, 26, 27, 28, 29 or 30 MBq, or any range between any two of the above values. However, as specified herein below, depending on the type of radiation emitted by the radionuclide and/or on the application, higher or lower doses may be envisaged.

The phrases “effective amount” or “therapeutically-effective amount” as used herein mean that amount of a compound, material, or composition comprising a compound of the invention, or other active ingredient which is effective for producing some desired therapeutic effect in at least a sub-population of cells in a patient at a reasonable benefit/risk ratio applicable to any medical treatment. A therapeutically effective amount with respect to a compound of the invention means that amount of therapeutic agent alone, or in combination with other therapies, that provides a therapeutic benefit in the treatment or prevention of a disease. Used in connection with a compound of the invention, the term can encompass an amount that improves overall therapy, reduces or avoids symptoms or causes of disease, or enhances the therapeutic efficacy of or synergies with another therapeutic agent.

According to a preferred embodiment, the radionuclide is a β-emitter as specified herein above, more preferably is ¹⁷⁷Lu and the use is diagnosis; in such case, the activity dosage of the complex preferably is at least 100 kBq/kg body weight, more preferably at least 500 kBq/kg body weight, most preferably at least 1 MBq/kg body weight. More preferably the radionuclide is a β-emitter as specified herein above, more preferably is ¹⁷⁷Lu and the use is therapy, preferably therapy of prostate carcinoma as specified elsewhere herein; in such case, the activity dosage of the complex preferably is at least 25 MBq/kg body weight, more preferably at least 50 MBq/kg body weight, most preferably at least 80 MBq/kg body weight. Thus, a preferred therapeutic dose in such case is of from 2 to 10 Gbq/patient, more preferably of from 4 to 8 GBq/patient, most preferably is about 6 GBq/patient.

More preferably, the radionuclide is an α-emitter as specified herein above, more preferably is ²²⁵AC and the use is therapy, preferably therapy of prostate carcinoma as specified elsewhere herein; in such case, the activity dosage of the complex is preferably in the range of from 25 kBq/kg to about 500 kBq/kg of body weight of said patient, more preferably, the activity dosage of the complex is at least 75 kBq/kg body weight, more preferably at least 100 kBq/kg body weight, still more preferably at least 150 kBq/kg body weight, most preferably at least 200 kBq/kg body weight. Thus, preferably, in such case, the activity dosage of the complex is of from 75 to 500 kBq/kg body weight, more preferably of from 100 to 400 kBq/kg body weight, still more preferably of from 150 to 350 kBq/kg body weight, most preferably of from 200 to 300 kBq/kg body weight.

The present invention also relates to a PSMA binding ligand as described above or below, a complex as described above or below, or a pharmaceutical composition as described herein above, for use in diagnosis, preferably for diagnosing a cell proliferative disease or disorder, in particular prostate cancer and/or metastases thereof. Further, the present invention also relates to a PSMA binding ligand as described above or below a complex as described above or below, or a pharmaceutical composition as described, for use in medicine, preferably for treating or preventing a cell proliferative disease or disorder, in particular prostate cancer and/or metastases thereof.

The term “diagnosing”, as used herein, refers to assessing whether a subject suffers from a disease or disorder, preferably cell proliferative disease or disorder, or not. As will be understood by those skilled in the art, such an assessment, although preferred to be, may usually not be correct for 100% of the investigated subjects. The term, however, requires that a, preferably statistically significant, portion of subjects can be correctly assessed and, thus, diagnosed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, or 0.05. As will be understood by the skilled person, diagnosing may comprise further diagnostic assessments, such as visual and/or manual inspection, determination of tumor biomarker concentrations in a sample of the subject, X-ray examination, and the like. The term includes individual diagnosis of as well as continuous monitoring of a patient. Monitoring, i.e. diagnosing the presence or absence of cell proliferative disease or the symptoms accompanying it at various time points, includes monitoring of patients known to suffer from cell proliferative disease as well as monitoring of subjects known to be at risk of developing cell proliferative disease. Furthermore, monitoring can also be used to determine whether a patient is treated successfully or whether at least symptoms of cell proliferative disease can be ameliorated over time by a certain therapy. Moreover, the term also includes classifying a subject according to a usual classification scheme, e.g. the T1 to T4 staging, which is known to the skilled person.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, as specified herein above. The term “preventing” and “prevention” refers to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the PSMA binding ligand according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed herein above.

Preferably, treatment and/or prevention comprises administration of at least one PSMA binding ligand and/or at least one complex as specified elsewhere herein, more preferably at an activity dosage and/or compound dosage as specified above.

The term “cell proliferative disease”, as used herein, relates to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body (metastasis). Preferably, also included by the term cancer is a relapse. Thus, preferably, the cancer is a solid cancer, a metastasis, or a relapse thereof. Preferably, the cell proliferative disease is an uncontrolled proliferation of cells comprising cells expressing PSMA.

Thus, preferably, the cell proliferative disease is a PSMA expressing cancer. The term “PSMA expressing cancer” refers to any cancer whose cancerous cells express Prostate Specific Membrane Antigen (PSMA). Preferably cancers (or cancer cells) that may be treated according to the invention are selected among prostate cancer, conventional renal cell cancers, cancers of the transitional cells of the bladder, lung cancers, testicular-embryonal cancers, neuroendocrine cancers, colon cancers, brain tumors and breast cancers, more preferably are selected among PSMA-positive prostate cancer, PSMA-positive renal cell cancers, PSMA-positive cancers of the transitional cells of the bladder, PSMA-positive lung cancers, PSMA-positive testicular-embryonal cancers, PSMA-positive neuroendocrine cancers, PSMA-positive colon cancers, PSMA-positive brain tumors, and PSMA-positive breast cancers. Whether a cancer is PSMA-positive can be established by the skilled person by methods known in the art, e.g. in vitro by immunostaining of a cancer sample, or in vivo e.g. by PSMA scintigraphy, preferably both as described in Kratochwil et al. (2017, J Nucl Med 58(10):1624. In particularly preferred aspects of the invention, said PSMA expressing cancer is prostate cancer or breast cancer, more preferably prostate cancer; and even more preferably advanced-stage prostate cancer. Thus, preferably, the cell proliferative disease is prostate cancer stage T2, more preferably stage T3, most preferably stage T4. Preferably, the cell proliferative disease is metastatic prostate cancer, more preferably is metastatic castration-resistant prostate cancer. Advantageously, it has been shown in the studies underlying the present invention that administration of the PSMA binding ligands and/or complexes of the present invention to a patient results in a reduced uptake of said PSMA binding ligands and/or complexes by the salivary and lacrimal glands, i.e. the patient's salivary and lacrimal glands, as compared to the uptake of e.g. the meanwhile commonly used PSMA-617. Due to the reduced uptake, adverse side effects on the salivary and/or lacrimal glands can be avoided and/or reduced. This is advantageous, because the adverse side effects on the salivary glands are considered as dosage-limiting (cf. Kratochwil et al. (2017, J Nucl Med 58(10):1624). Based on the finding of the present invention, larger amounts of PSMA binding ligands and/or complexes and in particular higher doses of radioactivity can be administered to a patient as compared to the PSMA binding ligands and complexes described in the art. Thus, the therapeutic window is broader than with the PSMA binding ligands presently in use. Also advantageously, the PSMA binding ligands of the present invention provide for improved diagnosis, since the co-labelling of irrelevant tissue and organs, in particular salivary glands, lacrimal glands and/or kidneys, is reduced.

Thus, the PSMA binding ligands and/or complexes of the present invention allow for the treatment of PSMA-expressing cancers, especially prostate cancer, and metastases thereof, and/or the diagnosis of PSMA-expressing cancers, especially prostate cancer, and metastases thereof, wherein adverse side effects on the patient's salivary glands and/or lacrimal glands are avoided and/or reduced. Thus, said treatment and/or diagnosis has less or less severe adverse side effects on the salivary glands and/or lacrimal glands or is preferably not accompanied by adverse side effects on the salivary glands and/or lacrimal glands at all. Preferably, the PSMA binding ligands of the present invention allow for reduction and/or avoidance of adverse side effects on the salivary glands and/or lacrimal glands while maintaining therapeutic efficacy essentially unchanged; thus, preferably, excretory properties of the PSMA binding ligands of the present invention are essentially unchanged compared to PSMA-617.

Accordingly, the PSMA binding ligands and/or complexes of the present invention allow for the treatment of PSMA-expressing cancers, especially prostate cancer, and metastases thereof, and/or the diagnosis of PSMA-expressing cancers, especially prostate cancer, and metastases thereof, wherein xerostomia is avoided.

Preferably, a PSMA binding ligands as described above or below, or a complex, as described above or below, or a pharmaceutical composition, are used for in vivo imaging and radiotherapy. Suitable pharmaceutical compositions may contain a radio imaging agent, or a radiotherapeutic agent that has a radionuclide either as an element, i.e. radioactive iodine, or a radioactive metal chelate complex of the compound of formula (la) and/or (lb) in an amount sufficient for imaging, together with a pharmaceutically acceptable radiological vehicle. The radiological vehicle should be suitable for injection or aspiration, such as human serum albumin; aqueous buffer solutions, e.g., tris(hydromethyl)-aminomethane (and its salts), phosphate, citrate, bicarbonate, etc; sterile water physiological saline; and balanced ionic solutions containing chloride and or dicarbonate salts or normal blood plasma cautions such as calcium potassium, sodium and magnesium.

The concentration of the imaging agent or the therapeutic agent in the radiological vehicle should be sufficient to provide satisfactory imaging. Appropriate dosages have been described herein above. The imaging agent or therapeutic agent should be administered so as to remain in the patient for about 1 hour to 10 days, although both longer and shorter time periods are acceptable. Therefore, convenient ampoules containing 1 to 10 mL of aqueous solution may be prepared.

Imaging may be carried out in a manner known to the skilled person, for example by injecting a sufficient amount of the imaging composition to provide adequate imaging and then scanning with a suitable imaging or scanning machine, such as a tomograph or gamma camera. In certain embodiments, a method of imaging a region in a patient includes the steps of: (i) administering to a patient a diagnostically effective amount of a compound complexed with a radionuclide; (ii) exposing a region of the patient to the scanning device; and (ii) obtaining an image of the region of the patient. In certain embodiments of the region imaged is the head or thorax. In other embodiments, the compounds and complexes of formula l(a) and/or (lb) target the PSMA protein.

Thus, in some embodiments, a method of imaging tissue such as spleen tissue, kidney tissue, or PSMA-expressing tumor tissue is provided including contacting the tissue with a complex synthesized by contacting a radionuclide and a formula (la) and/or formula (lb) compound.

The amount of the PSMA binding ligand of the present invention, or a formulation comprising a complex of the PSMA binding ligand, or its salt, solvate, stereoisomer, or tautomer that is administered to a patient depends on several physiological factors. These factors are known by the physician, including the nature of imaging to be carried out, tissue to be targeted for imaging or therapy and the body weight and medical history of the patient to be imaged or treated using a radiopharmaceutical.

Accordingly in another aspect, the invention provides a method for treating a patient by administering to a patient a therapeutically effective amount of a complex, as described above, to treat a patient suffering from a cell proliferative disease or disorder. Specifically, the cell proliferative disease or disorder to be treated or imaged using a compound, pharmaceutical composition or radiopharmaceutical in accordance with this invention is a cancer, for example, prostate cancer and/or prostate cancer metastasis in e.g. lung, liver, kidney, bones, brain, spinal cord, bladder, etc.

The compounds of the invention may e.g. be synthesized in solution as well as on solid phase using e.g. standard peptide coupling procedures, such as Fmoc solid phase or solution coupling procedures. Preferably, the chelator is coupled to the remaining part of the molecule in the last coupling step followed by a deprotection step and in case of solid phase chemistry, cleavage from the resin. However, other synthetic procedures are possible and known to the skilled person. A preferred synthesis of the compounds of the present invention is described in detail in the example section.

Summarizing the findings of the present invention, the following embodiments are preferred:
1. PSMA binding ligand, or a pharmaceutically acceptable salt or solvate thereof, comprising an oligosaccharide building block which comprises a bond being cleavable by alpha-amylase.
2. PSMA binding ligand according to embodiment 1, further comprising a PSMA binding motif Q and a chelator residue A.
3. PSMA binding ligand according to embodiment 2, wherein the PSMA binding motif Q and the chelator residue A are linked via at least one linker L^{AQ} comprising the oligosaccharide building block, the PSMA binding ligand preferably having the structure (I)

A-L^{AQ}-Q (I).
4. PMA binding ligand according to embodiment 2 or 3, wherein after cleavage of a bond present in the oligosaccharide building block by an alpha-amylase, the PSMA binding ligand is cleaved into at least two fragments, wherein one fragment comprises a PSMA binding motif Q and another fragment comprises the at least one chelator residue A.
5. PMA binding ligand according to embodiment 2 or 3, wherein the PSMA binding ligand is being cleavable by the alpha-amylase into at least two fragments, wherein one fragment comprises the PSMA binding motif Q and another fragment comprises the at least one chelator residue A
6. PSMA binding ligand according to any one of embodiments 1 to 5, having the structure (Ia)

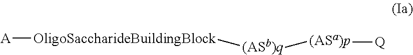

wherein Q is the PSMA binding motif, A is the chelator residue, AS^{a} and AS^{b} are amino acid building blocks, q is an integer of from 0-4, preferably 0-3, more preferably q is 1, and p is an integer of from 0 to 3, more preferably of from 1 to 3, more preferably p is 1.
7. PSMA binding ligand according to any one of embodiments 1 to 6, wherein the oligosaccharide building block comprises of from 2 to 10, preferably of from 3 to 10, more preferably of from 3 to 6 monosaccharide units, preferably 3 monosaccharide units.
8. PSMA binding ligand to any one of embodiments 1 to 7, wherein the oligosaccharide building block comprises a maltotriose moiety, wherein the maltotriose moiety preferably has a structure selected from structures (m1), (m2) and (m3):

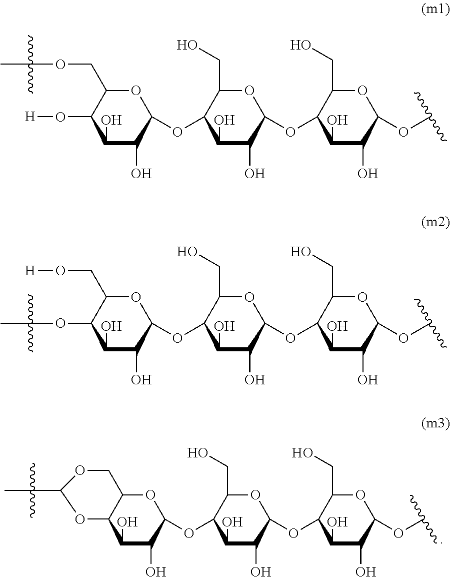

9. PSMA binding ligand according to any one of embodiments 1 to 7, wherein the ligand comprises a chelator residue A, A being derived from a chelator selected from the group consisting of 1,4,7,10-tetraazacyclododecane-N,N′,N″,N′″-tetraacetic acid (=DOTA), N,N″-bis[2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid] (NOPO), 3,6,9,15-tetraazabicyclo[9.3.1.]pentadeca-1(15),11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N-hydroxysuccinamide (DFO), Diethylenetriaminepentaacetic acid (DTPA), trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl -DTPA (MX-DTPA).
10. PSMA binding ligand according to embodiment 9, wherein A is a chelator residue having a structure selected from the group consisting of

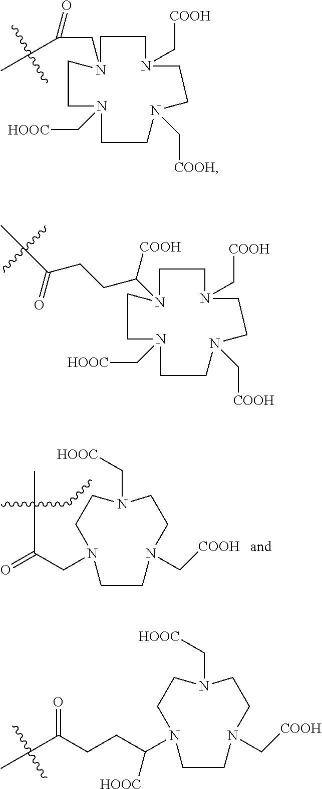

11. PSMA binding ligand according to any one of embodiments 1 to 10 having the structure

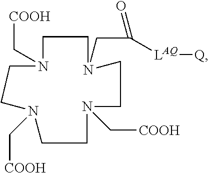

wherein Q is the PSMA binding motif and wherein L^{AQ} is a linker comprising the oligosaccharide building block.
12. PSMA binding ligand according to any one of embodiments 1 to 11 comprising a PSMA binding motif Q having the structure

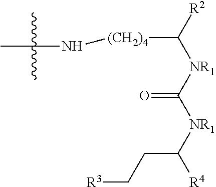

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂.
13. PSMA binding ligand according to any one of embodiments 1 to 12, comprising an amino acid building block AS^{a} having the structure

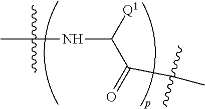

wherein Q¹ is selected from the group consisting of alkylaryl, arylalkyl, aryl, alkylheteroaryl, heteroarylalkyl and heteroaryl, and wherein p is an integer of from 0 to 3, preferably of from 1 to 3, more preferably p is 1.
14. PSMA binding ligand according to embodiment 13, wherein Q¹ comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably wherein Q¹ is selected from the group consisting of

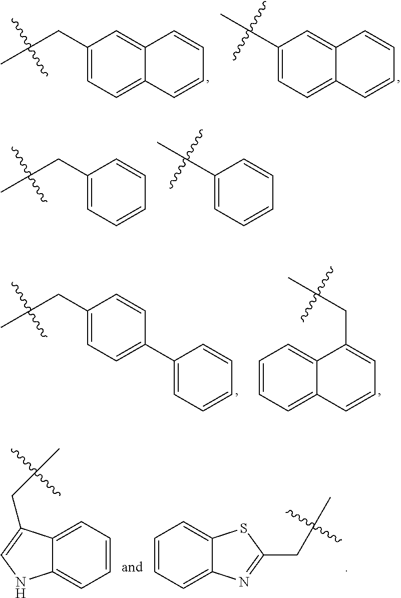

more preferably wherein Q¹ is

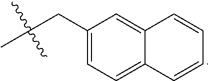 [SMILES: *Cc1ccc2ccccc2c1]

15. PSMA binding ligand according to any one of embodiments 1 to 14, comprising a PSMA binding motif having the structure

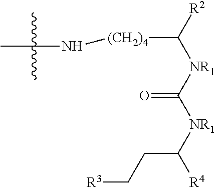

and an amino acid building block having AS^{a}

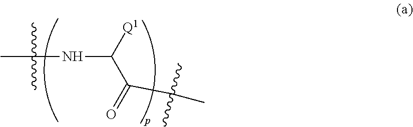

wherein the C-terminal end of the amino acid building block is attached to the_NH— group PSMA binding motif, and wherein p is 1, thereby forming the building block

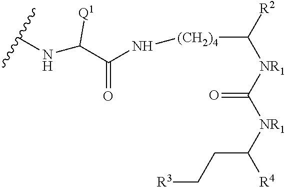

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂, and wherein Q¹ is selected from the group consisting of alkylaryl, arylalkyl, aryl, alkylheteroaryl, heteroarylalkyl and heteroaryl, wherein Q¹ preferably comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably wherein Q¹ is selected from the group consisting of

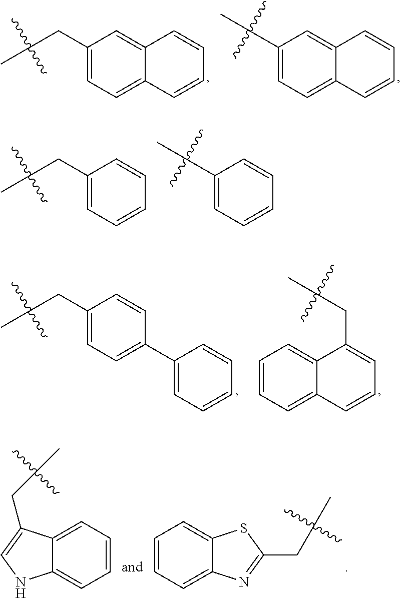

more preferably wherein Q¹ is

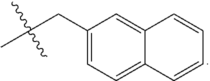 [SMILES: *Cc1ccc2ccccc2c1]

16. PSMA binding ligand according to any one of embodiments 1 to 15, comprising an amino acid building block AS^{b} having the structure (b)

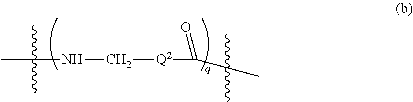

wherein Q² is selected from the group consisting of aryl, alkylaryl, arylalkyl, cycloalkyl, heterocycloalkyl, heteroaryl, heteroarylalkyl and alkylheteroaryl, and wherein q is an integer of from 0-4, preferably 1, wherein Q² is preferably

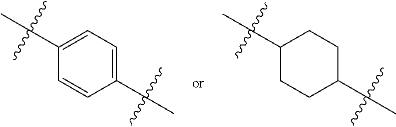 [SMILES: *c1ccc(*)cc1 ; *C1CCC(*)CC1]

preferably

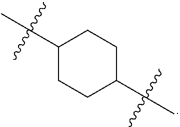 [SMILES: *C1CCC(*)CC1]

17. PSMA binding ligand according to any one of embodiment 16, wherein the oligosaccharide building block is being attached to the N terminal end of AS^{b}.
18. PSMA binding ligand according to any one of embodiments 1 to 17, wherein the oligosaccharide building block is being attached to a carbonyl group of the chelator residue A.
19. PSMA binding ligand according to any one of embodiments 1 to 18, wherein the ligand further comprises a building block having the structure ((X¹)ₙ₁(X²)ₙ₂)ₙ, wherein X¹ and X² are independently of each other, charged amino acids, n is of from 0 to 9, and n1 and n2, are independently of each other, an integer of from 0 to 3.
20. PSMA binding ligand according to any one embodiments 1 to 19, wherein the ligand further comprises a (EH)₃ building block.
21. PSMA binding ligand according to embodiments 1 to 20, wherein the oligosaccharide building block has the structure (c)

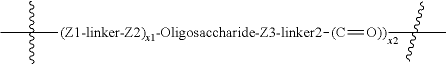

wherein Z1, Z2 and Z3 are functional groups and linker1 and linker2 are linking moieties, x1 and x2 are integers which are, independently of each other, 0, 1 or 2, preferably 0 or 1, more preferably both 1.
22. PSMA binding ligand according embodiment 21, wherein the oligosaccharide building block has the structure

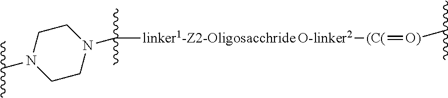

23. PSMA binding ligand according embodiment 21, wherein the oligosaccharide building block has the structure

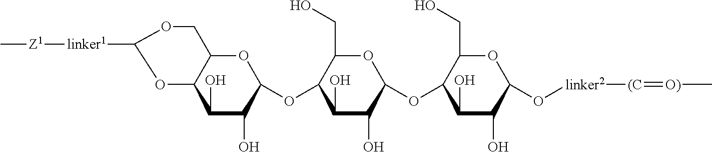

24. PSMA binding ligand according embodiment 21, wherein the oligosaccharide building block has the structure

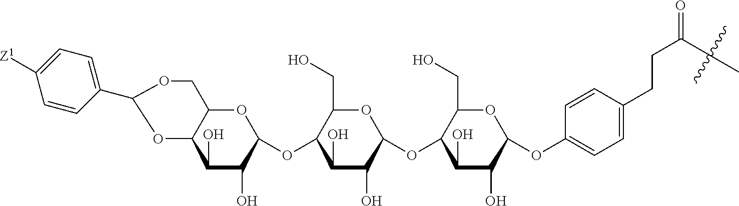

25. PSMA binding ligand according embodiment 21, wherein the oligosaccharide building block has the structure

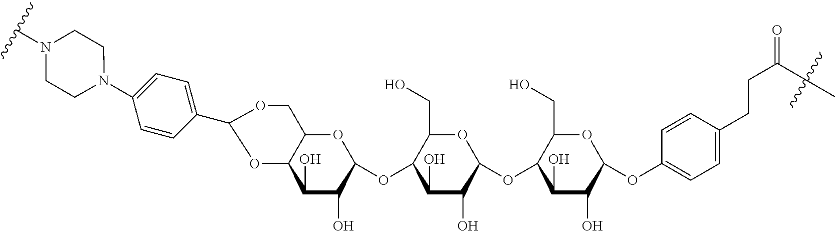

26. PSMA binding ligand according to formula (Ia)

A−OligoSaccharideBuildingBlock−(AS^{b})q−(AS^{a})p·Q (Ia)

or a pharmaceutically acceptable salt or solvate thereof, wherein Q is a PSMA binding motif, A is a chelator residue, AS^{a} and AS^{b} are amino acid building blocks and q is an integer of from 0 to 4 and p is an integer of from 0 to 3.
27. PSMA binding ligand according to embodiment 26, with AS^{a} having the structure

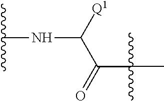

wherein Q¹ is selected from the group consisting of alkylaryl, arylalkyl, aryl, alkylheteroaryl, heteroarylalkyl and heteroaryl,
28. PSMA binding ligand according to embodiment 27, wherein Q¹ comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably wherein Q¹ is selected from the group consisting of

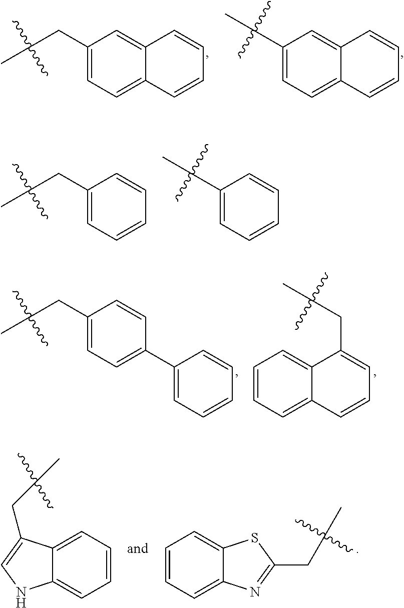

more preferably wherein Q¹ is

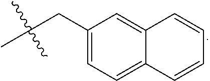 [SMILES: *Cc1ccc2ccccc2c1]

29. PSMA binding ligand according to any one of embodiments 26 to 28, wherein Q has the structure

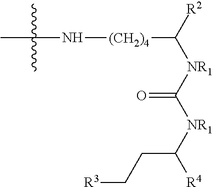

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂, and wherein Q¹ is selected from the group consisting of alkylaryl, arylalkyl, aryl, alkylheteroaryl, heteroarylalkyl and heteroaryl, wherein Q¹ preferably comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably wherein Q¹ is selected from the group consisting of

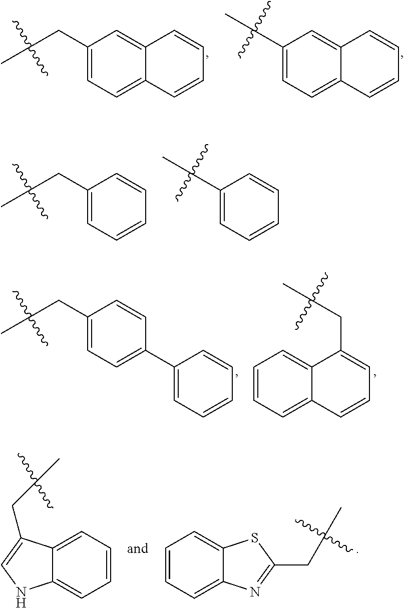

more preferably wherein Q¹ is

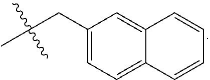 [SMILES: *Cc1ccc2ccccc2c1]

30. PSMA binding ligand according to any one of embodiments 26 to 29, wherein AS^{b} has the structure (b)

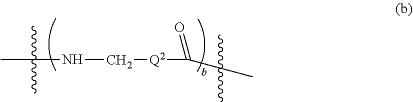

wherein Q² is selected from the group consisting of aryl, alkylaryl, arylalkyl, cycloalkyl, heterocycloalkyl, heteroaryl, heteroarylalkyl and alkylheteroaryl, and wherein q is an integer of from 0-4, preferably 1, wherein Q² is preferably

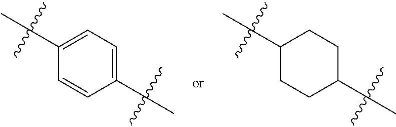 [SMILES: *c1ccc(*)cc1 ; *C1CCC(*)CC1]

preferably

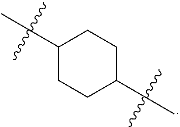 [SMILES: *C1CCC(*)CC1]

31. PSMA binding ligand according to any one of embodiments 26 to 30, wherein A is a chelator residue derived from a chelator selected from the group consisting of 1,4,7,10-tetraazacyclododecane-N,N′,N″,N′″-tetraacetic acid (=DOTA), N,N″-bis[2-hydroxy-5-(carboxyethyl)benzyl]ethylenediamine-N,N″-diacetic acid, 1,4,7-triazacyclononane -1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid] (NOPO), 3,6,9,15-tetraazabicyclo[9.3.1.]pentadeca -1(15),11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N -hydroxysuccinamide (DFO), Diethylenetriaminepentaacetic acid (DTPA), Trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA).
32. PSMA binding ligand according to any one of embodiments 26 to 31, wherein A is a chelator residue having a structure selected from the group consisting of

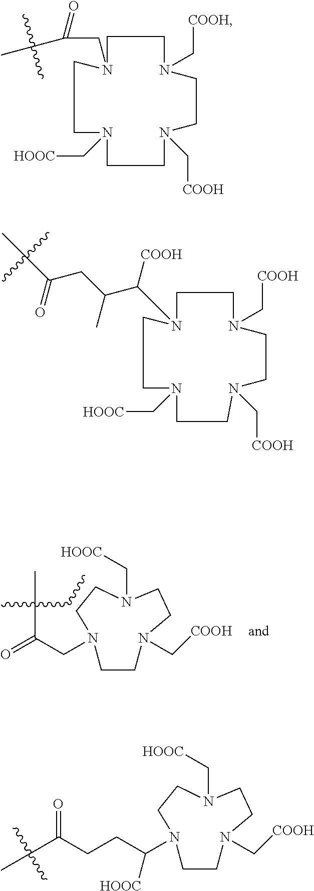

33. PSMA binding ligand according to any one of embodiments 26 to 31, wherein the oligosaccharide building block has the structure (c)

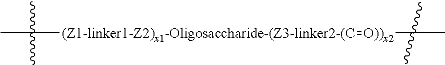

wherein Z1, Z2 and Z3 are functional groups and linker1 and linker2 are linking moieties, x1 and x2 are integers which are, independently of each other, 0, 1 or 2, preferably 0 or 1, more preferably both 1.
34. PSMA binding ligand according to embodiment 33, wherein the oligosaccharide building block has the structure

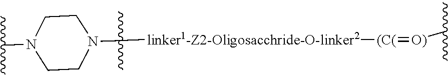

35. PSMA binding ligand according to embodiment 33, wherein the oligosaccharide building block has the structure

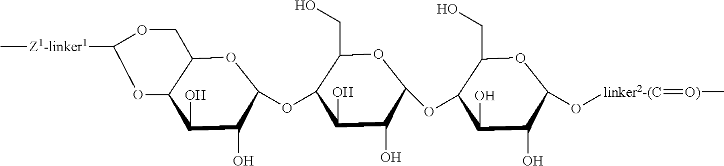

36. PSMA binding ligand according to embodiment 33, wherein the oligosaccharide building block has the structure

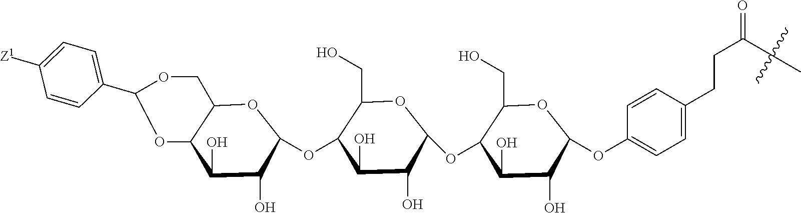

37. PSMA binding ligand according to embodiment 33, wherein the oligosaccharide building block has the structure

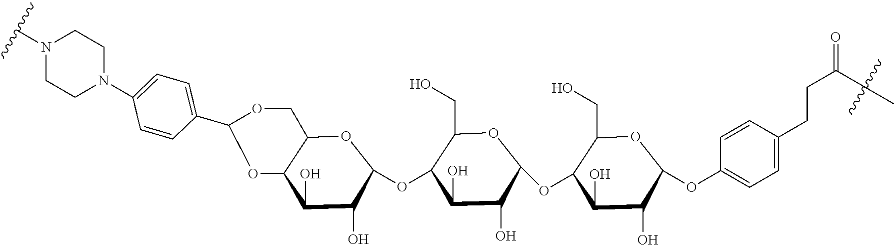

38. PSMA binding ligand of formula (II)

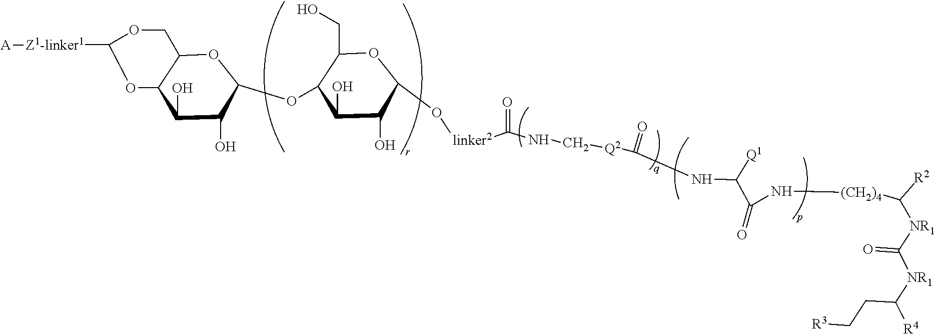

or a pharmaceutically acceptable salt or solvate thereof, wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂. Q¹ is selected from the group consisting of alkylaryl, arylalkyl, aryl, alkylheteroaryl, heteroarylalkyl and heteroaryl, Q² is selected from the group consisting of aryl, alkylaryl, arylalkyl, cycloalkyl, heterocycloalkyl, heteroaryl, heteroarylalkyl and alkylheteroaryl, A is a chelator residue derived from a chelator selected from the group consisting of 1,4,7,10-tetraazacyclododecane-N,N′,N″,N″′-tetraacetic acid (=DOTA), N,N″-bis[2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid] (NOPO), 3,6,9,15-tetraazabicyclo[9.3.1]pentadeca -1(15),11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N -hydroxysuccinamide (DFO), Diethylenetriaminepentaacetic acid (DTPA), Trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p -isothiocyanatobenzyl)-4-methyl-DTPA (1 M3B) and 1-(2)-methyl-4-isocyanatobenzyl -DTPA (MX-DTPA), q is an integer of from 0-4, p is an integer of from 0 to 3, r is an integer from 1 to 10, preferably 2-5, Z1 is a functional moiety being attached to a carbonyl group of A and linker1 and linker2 are, independently selected from the group consisting of alkyl, alkenyl, alkynyl, alkylaryl, arylalkyl, aryl, alkylarylalkyl, heteroaryl, alkylheteroaryl, alkylheteroalkyl, heteroarylalkyl, cycloalkyl, cycloheteroalkyl and peptidic groups.
39. The PSMA binding ligand of embodiment 38, wherein A is a chelator residue having a structure selected from the group consisting of

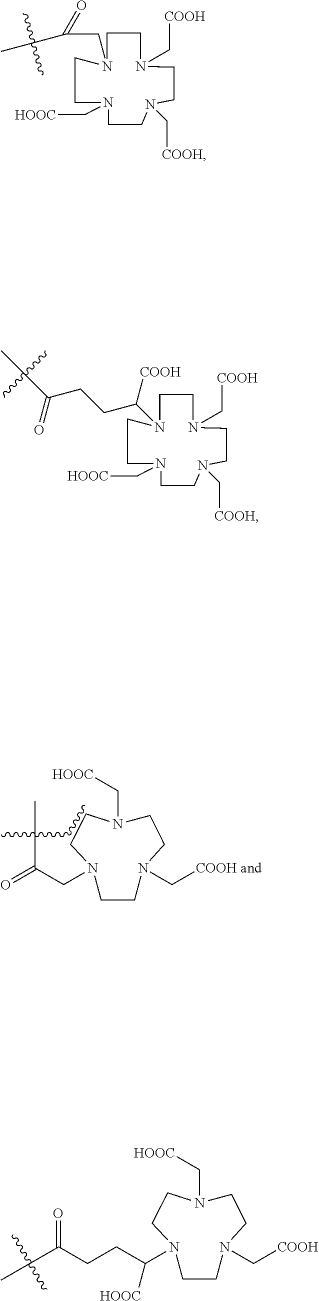

40. The PSMA binding ligand of embodiment 38 or 39, wherein Q¹ preferably comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably wherein Q¹ is selected from the group consisting of

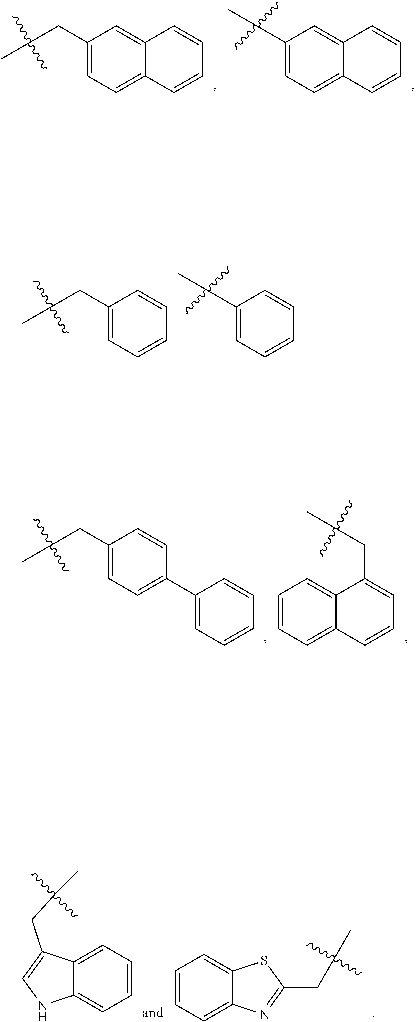

preferably wherein Q¹ is

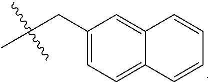 [SMILES: *Cc1ccc2ccccc2c1]

41. The PSMA binding ligand according to any one of embodiments 38 to 40, wherein R³, R² and R⁴ are —CO₂H and R¹ is H.
42. The PSMA binding ligand according to any one embodiments 38 to 40, wherein Q² is

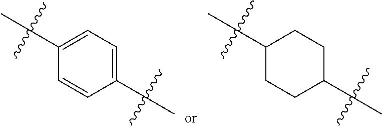 [SMILES: *c1ccc(*)cc1 ; *C1CCC(*)CC1]

preferably

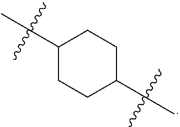 [SMILES: *C1CCC(*)CC1]

43. The PSMA binding ligand according to any one of embodiments 26 to 42, wherein linker1 is an aryl group, preferably a phenyl group
44. The PSMA binding ligand according to any one of embodiments 26 to 42, wherein linker2 is an arylalkyl group.
45. Complex comprising
   (a) a radionuclide, and
   (b) the PSMA binding ligand according to any one of embodiments 1 to 44 or a pharmaceutically acceptable salt or solvate thereof.
46. The complex of embodiment 45, wherein, the radionuclide is selected from the group consisting ⁸⁹Zr, ⁴⁴Sc, ¹¹¹ln, ⁹⁰Y, ⁶⁶Ga, ⁶⁷Ga, ⁶⁸Ga, ¹⁷⁷Lu, ^{99m}Tc, ⁶⁰Cu, ⁶¹Cu, ⁶²Cu, ⁶⁴Cu, ⁶⁶Cu, ⁶⁷Cu, ¹⁴⁹Tb, ¹⁵²Tb, ¹⁵⁵Tb, ¹⁵³Sm, ¹⁶¹Tb, ¹⁵³Gd, ¹⁵⁵Gd, ¹⁵⁷Gd, ²¹³Bi, ²²⁵Ac, ²³⁰U, ²²³Ra, ¹⁶⁵Er, ⁵²Fe, ⁵⁹Fe, and radionuclides of Pb (such as ²⁰³Pb and ²¹²Pb, ²¹¹Pb, ²¹³Pb, 214Pb, ²⁰⁹Pb, ¹⁹⁸Pb, ¹⁹⁷Pb).
47. A pharmaceutical composition comprising the PSMA binding ligand of any one of embodiments 1 to 44 or a complex of embodiment 45 or 46.
48. The PSMA binding ligand of any one of embodiments 1 to 44 or a complex of embodiment 45 or 46 or a pharmaceutical composition of embodiment 47 for use in medicine, preferably for treating and/or preventing prostate cancer and/or metastases thereof.
49. The the PSMA binding ligand of embodiment 48, wherein adverse side effects on the salivary glands and/or lacrimal glands are reduced and/or avoided.
50. PSMA binding ligand of any one of embodiments 1 to 44 or a complex of embodiment 45 or 46 or a pharmaceutical composition of embodiment 47 for use in diagnostics.
51. PSMA binding ligand of any one of embodiments 1 to 44 or a complex of embodiment 45 or 46 or a pharmaceutical composition of embodiment 47 in the diagnosis of cancer, in particular of prostate cancer and/or metastases thereof.

The following examples shall merely illustrate the invention. Whatsoever, they shall not be construed as limiting the scope of the invention.

### FIGURES

FIG. 1. The time-dependent cleavability of the compound PSMA-MT by α-amylase is shown. ¹⁷⁷Lu-PSMA-MT was incubated with an α-amylase (isolated from the human salivary gland) and enzymatic cleavage was monitored at different times by analytical HPLC.

FIG. 2: μPET imaging with ^{Ga}-labeled PSMA-MT.

FIG. 3: Internalization of PSMA-MT in comparison to PSMA-617

FIG. 4: Organ distribution of PSMA-MT in comparison to PSMA-617 (¹⁷⁷Lu-labeled)

FIG. 5: Chemical Formula of PSMA-MT with potential alpha-amylase cleavage sites A and B indicated, as well as cleavage products

### EXAMPLES

All commercially available chemicals were of analytical grade and used without further purification. [¹⁷⁷Lu]LuCl₃ was obtained from ITG. The compounds were analyzed using reversed-phase high performance liquid chromatography (RP-HPLC; Chromolith RP-18e, 100×4.6 mm; Merck, Darmstadt, Germany). Analytical HPLC runs were performed using a linear gradient 5% (A) (0.1% aqueous TFA) to 50% B (0.1% TFA in CH₃CN)) in 24 min at 1 mL/min.

Analytical HPLC runs were performed using the system Agilent 1200 series (Agilent Technologies, Santa Clara, Calif., USA). UV absorbance was measured at 220 and 280 nm, respectively. For mass spectrometry a LC-MS SQ300 (Perkin Elmer, Waltham, Mass., USA) was used.

The precursor PSMA-617 (2-[3-(1-Carboxy-5-{3-naphthalen-2-yl-2-[(4-{[2-(4,7,10-tris-carboxymethyl-1,4,7,10-tetraaza-cyclododec-1-yl)-acetylamino]-methyl}-cyclohexanecarbonyl) -amino]-propionylamino}-pentyl)-ureido]-pentanedioic acid) was purchased from ABX, Radeberg, Germany.

### Example 1: Synthesis of “PSMA-MT”

### 1-Fmoc-4-(3-(diethoxymethyl)phenyl)piperazine

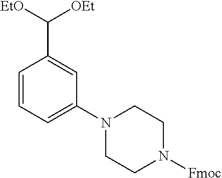

A suspension of 648 mg (2.50 mmol) 1-bromo-3-(diethoxymethyl)benzene, 646 mg (7.50 mmol) Piperazine, 280 mg (2.50 mmol) potassium tert-butoxide, 18.7 mg (30.0 μmol) rac-BINAP and 11.5 mg (12.5 μmol) Tris(dibenzylideneacetone)dipalladium(0) in 5 mL dry dioxane is heated to 60° C. over 16 h under N₂. After cooling to room temperature, the red suspension is filtered over a short pad of SiO₂ (ca. 5 g) and eluted with 100 mL of ethyl acetate containing 1% triethylamine. 843 mg (2.50 mmol) Fmoc N-hydroxysuccinimide ester are directly added to the filtrate and left stirring at room temperature for 2 h. After completion the solvent is removed, the residue taken up in dichloromethane and purified by column chromatography (50 g SiO₂; hexanes/ethyl acetate 4:1). After evaporation, 778 mg (1.60 mmol; 64%) of the desired product are obtained.

R_{f}(hexanes/ethyl acetate 2:1)=0.35

### 1-(4-(3-allyloxy-3-oxopropyl)phenyl)decaacetylmaltotriose

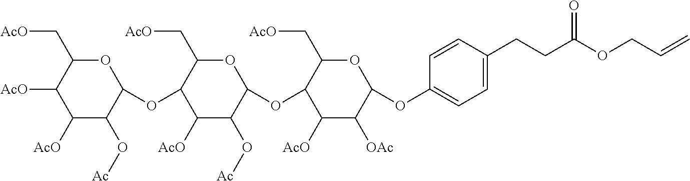 [SMILES: C=CCOC(=O)CCc1ccc(OC2OC(COC(C)=O)C(OC3OC(COC(C)=O)C(OC4OC(COC(C)=O)C(OC(C)=O)C(OC(C)=O)C4OC(C)=O)C(OC(C)=O)C3OC(C)=O)C(OC(C)=O)C2OC(C)=O)cc1]

500 mg (517 μmol) undecaacetylmaltotriose and 200 mg (1.04 mmol) ally 3-(4-hydroxyphenyl)propanoate[1] are dissolved in 20 mL dichloromethane. The solution is cooled to 0° C. and 185 μL (213 mg; 1.50 mmol) boron trifluoride diethyl etherate is added dropwise under an inert atmosphere. The reaction is allowed to reach room temperature and stirred under reflux for 24 h. The reaction mixture is poured to 50 g ice and stirred for 30 min. The layers are separated and the water phase is extracted with dichloromethane (3×15 mL). The organic phases are washed with sat. sodium bicarbonate and brine and dried over magnesium sulfate. After evaporation the residue is filtrated over SiO₂ (10 g; 120 mL hexanes/ethyl acetate 1:1) and purified by RP-HPLC (50-70% acetonitrile over 25 min). After freeze drying 305 mg (274 μmol; 53%) of the desired trisaccharide are obtained.

### 1-(4-(3-carboxypropyl)phenyl)maltotriose

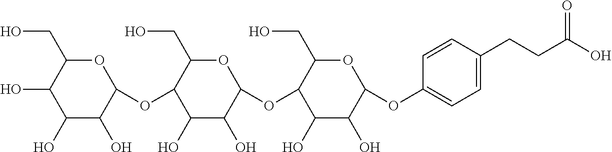 [SMILES: O=C(O)CCc1ccc(OC2OC(CO)C(OC3OC(CO)C(OC4OC(CO)C(O)C(O)C4O)C(O)C3O)C(O)C2O)cc1]

305 mg (274 μmol) of lyophilized 1-(4-(3-allyloxy-3-oxopropyl)phenyl)decaacetylmaltotriose are dissolved in 10 mL tetrahydrofuran/water 6:4 and stirred with 100 mg lithium hydroxide overnight. After completion Tetrahydrofuran is removed and the resulting solution purified by RP-HPLC (5-20% acetonitrile in 25 min). After freeze drying 135 mg (207 μmol; 75%) of the desired product are obtained.

### 1-(4-(3-carboxypropyl)phenyl)-4″,6″-(3-(4-Fmoc-piperazine)benzyliclene)maltotriose

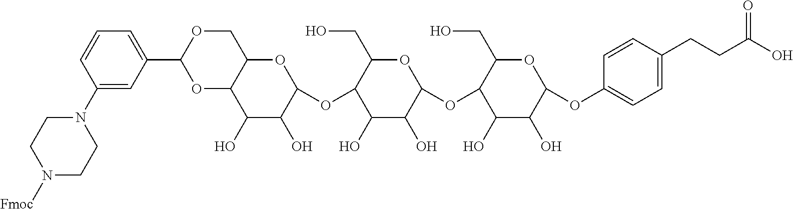

9.60 mg (14.7 μmol) 1-(4-(3-carboxypropyl)phenyl)maltotriose, 27.8 mg (57.1 μmol) and 3.51 mg (18.4 μmol) p-toluenesulfonic acid monohydrate are dissolved in 100 μL dimethylformamide and heated to 60° C. for 1 h. The rection mixture is directly purified by RP-HPLC (20-50% acetonitrile in 25 min) yielding 6.76 mg (6.46 μmol; 44%) of the desired compound after freeze drying.

### 1-(4-(3-oxo-3-PSMA-propyl)phenyl)-4″,6″-(3-(4-Fmoc-piperazine)benzyliclene)maltotriose

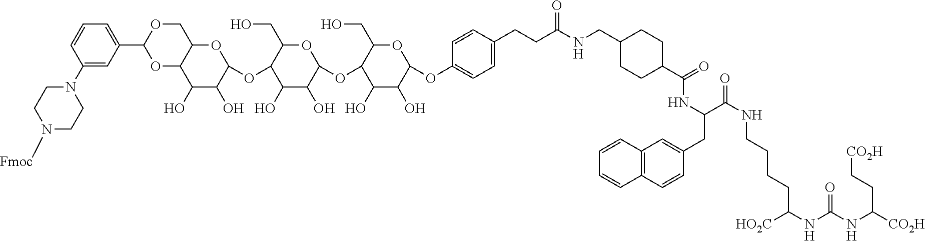

1.94 mg (6.43 mmol) N,N,N′,N′-Tetramethyl-O-(N-succinimidyl)uronium tetrafluoroborate (TSTU) are added to 5.61 mg (5.36 μmol) 1-(4-(3-carboxypropyl)phenyl)-4″,6″-(3-(4-Fmoc -piperazine)benzylidene)maltotriose and 1.23 mg (10.7 μmol) N-hydroxysuccinimide in 95 dimethylformamide and 5 μL N,N-diisopropylethylamine. The solution is left for 15 min until 5.27 mg (8.03 μmol) of the PSMA building block (((1R)-5-(2-(4-(aminomethyl)cyclohexane-1-carboxamido)-3-(naphthalene-2-yl)propanamido)-1-carboxypentyl)carbamoyl)-L-glutamic acid, (obtained following Benesova et al. For this purpose the resin bound compound (8) in doi: 10.2967/jnumed.114.147413 was cleaved from the resin: After Fmoc-deprotection of the trans-1,4-aminomethylcyclohexylcarboxamide residue, the compound was cleaved with 95% TFA (2.5% TIS, 2.5% water) and purified by HPLC followed by freeze drying.) are added. Another 95 dimethylformamide and 5 μL N,N-diisopropylethylamine are added to the turbid solution, which is then heated to 45° C. until the solution clears up. The reaction mixture was quenched with 500 acetonitrile/water 1:1 and purified by RP-HPLC (20-50% acetonitrile in 25 min). The fraction containing the desired product was evaporated to dryness and directly used for the following step.

### 1-(4-(3-oxo-3-PSMA-propyl)phenyl)-4″,6″-(3-(4-DOTA-piperazine)benzylidene)maltotriose (PSMA-MT)

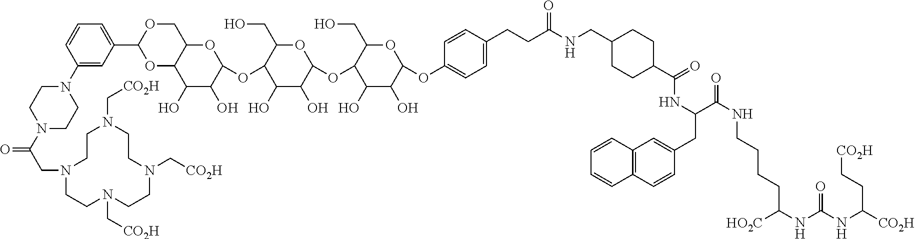 [SMILES: O=C(O)CCC(NC(=O)NC(CCCCNC(=O)C(Cc1ccc2ccccc2c1)NC(=O)C1CCC(CNC(=O)CCc2ccc(OC3OC(CO)C(OC4OC(CO)C(OC5OC6COC(c7cccc(N8CCN(C(=O)CN9CCN(CC(=O)O)CCN(CC(=O)O)CCN(CC(=O)O)CC9)CC8)c7)OC6C(O)C5O)C(O)C4O)C(O)C3O)cc2)CC1)C(=O)O)C(=O)O]

The residue obtained in the previous step (1-(4-(3-oxo-3-PSMA-propyl)phenyl)-4″,6″-(3-(4-Fmoc-piperazine)benzylidene)maltotriose) is taken up in 4 mL of dimethylformamide/piperidine 4:1 and left standing for 5 min. After thorough evaporation of the solvent 10.0 mg (19.0 μmol) DOTA-p-nitrophenylester, 4 mL dimethylformamide and 40 μL N,N-diisopropylethylamine are added. After 2 h, the solvent is removed and the residue taken up in 2 mL acetonitrile/water 1:1 before purification via RP-HPLC (10-30% acetonitrile in 25 min). 3.31 mg (1.79 μmol; 33%) of the final product are obtained after freeze drying.
1. M.-K. Lee, Y. B. Park, S.-S. Moon, S. H. Bok, D.-J. Kim, T.-Y. Ha, T.-S. Jeong, K.-S. Jeong, M.-S. Choi, Chemico-Biological Interactions 2007, 170, 9-19.

### Example 1A: Radiolabeling with ¹⁷⁷Lu

The γ- and β-emitter [¹⁷⁷Lu]LuCl₃ (ITG, Munich), with a t_{1/2} of 6.7 days was used. Radiolabeling was performed by adding 13 μL [¹⁷⁷Lu]LuCl₃ (˜30-37 MBq) in 0.4 mM HCl, 7 μL of diluted compound (0.1 mM solution of PSMA-MT in nanopure H₂O) to 230 μL ammonium-acetate buffer (0.5 M, pH 5.4). The reaction mixture was incubated at 95° C. for 30 min to yield ¹⁷⁷Lu-PSMA-MT. The radiochemical yield (RCY) was determined using high performance liquid chromatography (RP-HPLC; Chromolith RP-18e, 100×4.6 mm; Merck, Darmstadt, Germany). Analytical HPLC runs were performed using an Agilent 1200 series (Agilent Technologies, Santa Clara, Calif., USA) equipped with a γ-detector. HPLC runs were performed using a linear gradient of A (0.1% trifluoroacetic acid (TFA) in water) to B (0.1% TFA in acetonitrile) (gradient: 5% B to 80% B in 15 min) at a flow rate of 2 mL/min.

### Example 1B: Radiolabeling with ⁶⁸Ga

⁶⁸Ga (half-life 68 min; β⁺ 89%; E_{β+} max. 1.9 MeV) was obtained from an in house ⁶⁸Ge/⁶⁸Ga generator (DKFZ Heidelberg, Germany) based on pyrogallol resin support. Approximately 0.5-1 GBq ⁶⁸Ga was eluted using 5.5 M HCl. The activity was trapped on a small anion-exchanger cartridge (AG1×8, Biorad, Richmond, Calif., USA) as [⁶⁸Ga]GaCl₄⁻. The radiogallium was eluted from the cartridge in a final volume of 300 μL ultrapure water (Merck, Darmstadt, Germany) as [⁶⁸Ga]GaCl₃. The precursor peptides (1 nmol in 2.4 M HEPES buffer, 90 μL) were added to 40 μL [⁶⁸Ga]Ga³⁺ eluate (˜40 MBq). The pH was adjusted to 4.2 using 30% NaOH and 10% NaOH. The reaction mixture was incubated at 98° C. for 10 minutes. The radiochemical yield (RCY) was determined by HPLC.

### Example 2: Cleavability of the Linker

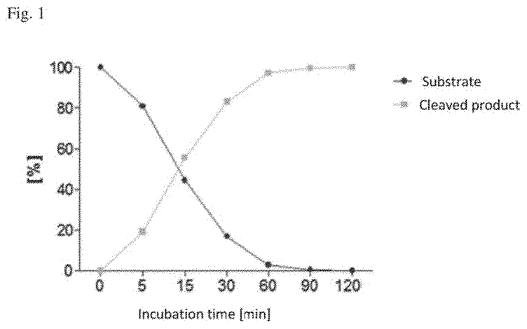

The basic cleavability of the linker by α-amylases was demonstrated after radioactive labeling of PSMA-MT in vitro according to example 1A (FIG. 1). The radiolabelled compound was incubated with α-amylase (isolated from the human salivary gland, Sigma-Aldrich) according to instructions of the manufacturer and enzymatic cleavage was monitored at different times by analytical HPLC (see FIG. 1).

### Example 3: μPET-Imaging

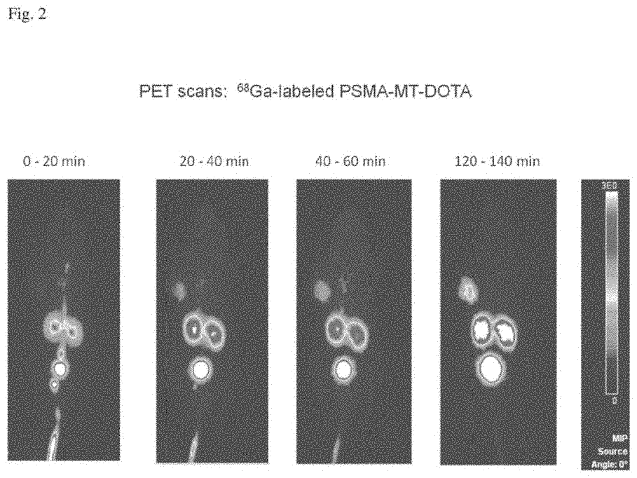

For μPET imaging, mice bearing a PSMA positive tumor were anaesthetized (2% sevoflurane, Abbott), placed into a small animal PET scanner (Inveon PET, Siemens) and injected with ⁶⁸Ga-labeled PSMA-MT (see example 1B). A 20 min transmission scan, a 50 min dynamic scan and a static scan from 100 to 120 min p.i. were performed. Images were reconstructed iteratively using the space alternating generalized expectation maximization method (SAGE, 16 subsets, 4 iterations) applying median root prior correction and were converted to standardized uptake value (SUV) shown in maximum intensity projection (MIP) images. Quantitation was done using a ROI (region of interest) technique and expressed as SUVmean (see FIG. 2).

### Example 4: Competitive Cell Binding

For the determination of competitive cell binding the cells (10⁵ per well) were incubated with a 0.8 nM solution of ⁶⁸Ga-labeled radioligand [Glu-urea-Lys(Ahx)]₂-HBED-CC (PSMA-10, precursor ordered from ABX, Radeberg, Germany) in the presence of 12 different concentrations of analyte (non-labeled compounds, 0-5000 nM, 100 μL/well). After incubation, the mixture was removed and the wells were washed 3 times with PBS using a multiscreen vacuum manifold (Millipore, Billerica, Mass.). Cell-bound radioactivity was measured using a gamma counter (Packard Cobra II, GMI, Minn., USA). The 50% inhibitory concentration (IC50) values were calculated by fitting the data using a nonlinear regression algorithm (GraphPad Software).

In first preliminary experiments for PSMA-MT an IC₅₀ of around 39 nM was determined, which is in the same range as the IC₅₀ of PSMA-617 (ABX, Radeberg, Germany) which was detected to be around 20 nM.

### Example 5: Internalization of PSMA-MT in Comparison to PSMA-617

10⁵ LNCaP cells were seeded in poly-L-lysine coated 24-well cell culture plates. 24 h later, wells were washed and the cells incubated with a 30 nM solution of ¹⁷⁷Lu radiolabeled PSMA-ligand (PSMA-617 or PSMA-MT) in 250 μL medium, for 45 min at 37° C. In the blocking experiments, incubation was performed in the presence of excess of 2-PMPA to assess competition for PSMA. Following incubation, PSMA-ligand containing medium was removed by washing 3 times with 1 mL of ice-cold PBS. Cells were subsequently incubated twice with 0.5 mL glycine-HCl in PBS (50 mM, pH=2.8) for 5 min to extract the cell membrane associated fraction. The cells were then washed with 1 mL of ice-cold PBS, and lysed with 0.5 mL 0.3 M NaOH. The membrane bound and lysed (internalized) fractions were assessed for radioactivity levels using a gamma counter (Packard Cobra II, GMI, Minn., USA). Cell uptake was calculated as percent of the initially added radioactivity detected in 10⁵ cells [% IA/10⁵ cells].

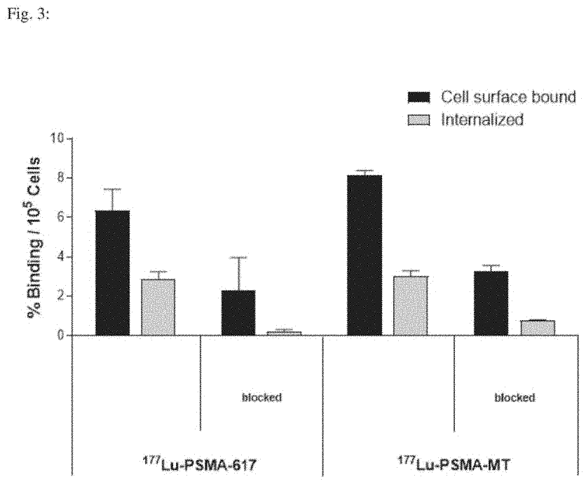

The results are given in the following table (see also FIG. 3):

| | | |
|---|---|---|
| 37° C. | Mean | Standard Deviation |

177Lu-PSMA-MT

| | | |
|---|---|---|
| Specifically internalized | 2,14459836 | 0,24994955 |
| Specifically cell surface bound | 4,86794251 | 0,56488708 |

177Lu-PSMA-617

| | | |
|---|---|---|
| Specifically internalized | 2,63598315 | 0,51667575 |
| Specifically cell surface bound | 4,09228993 | 1,14873804 |

### Example 6: Organ Distribution of PSMA-MT in Comparison to PSMA-617 (¹⁷⁷Lu-labeled)

5×10⁶ LNCaP cells were subcutaneously inoculated into the right flank of male 6-week-old BALB/c nu/nu mice (Charles River Laboratories). Tumors were grown for ˜3 weeks, to ˜200 mm³. 60 pmoles of ¹⁷⁷Lu labelled PSMA-ligands were dissolved in 100 μL 0.9% NaCl and injected per mouse. The solution was injected via the tail vein, followed by sacrifice at various time points. Organs of interest (blood, heart, lung, spleen, liver, kidney, muscle, small intestine, brain, tumor, and femur) were dissected, blotted dry, and weighed. The radioactivity was measured using a gamma counter and calculated as % ID/g, and corrected for ¹⁷⁷Lu decay.

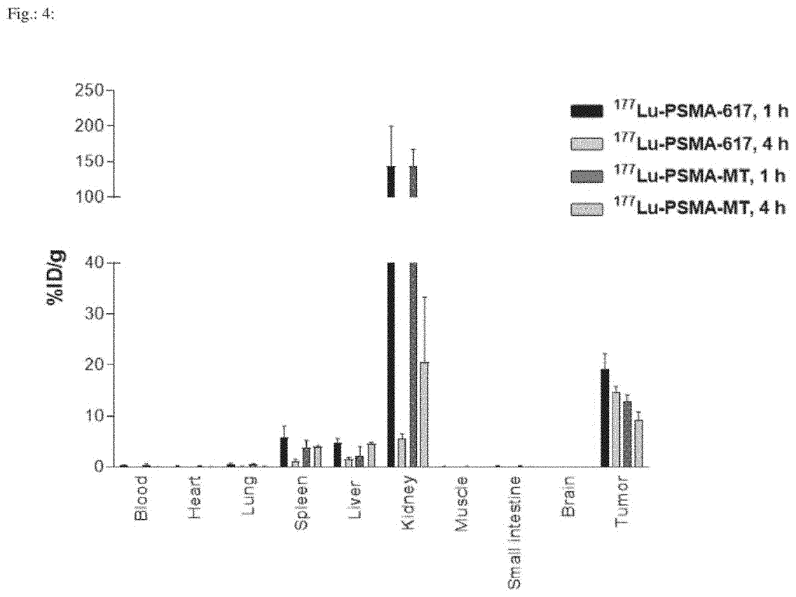

The results are given in the following tables (mean+standard deviation; per time point n=3 animals) (see also FIG. 4):

¹⁷⁷Lu-PSMA-617: 1 h

| | | | | | | | Small | | | | |
|---|---|---|---|---|---|---|---|---|---|---|---|
| Blood | Heart | Lung | Spleen | Liver | Kidney | Muscle | Intestine | Brain | Tumor | Kidney | Tail |
| 0.31 | 0.17 | 0.61 | 5.86 | 4.73 | 143.55 | 0.07 | 0.18 | 0.02 | 19.20 | 146.77 | 3.46 |
| 0.10 | 0.05 | 0.12 | 2.21 | 0.90 | 56.58 | 0.03 | 0.07 | 0.00 | 3.01 | 57.40 | 2.25 |

¹⁷⁷Lu-PSMA-617: 4 h

| | | | | | | | Small | | | | |
|---|---|---|---|---|---|---|---|---|---|---|---|
| Blood | Heart | Lung | Spleen | Liver | Kidney | Muscle | Intestine | Brain | Tumor | Kidney | Tail |
| 0.03 | 0.03 | 0.08 | 1.20 | 1.57 | 5.52 | 0.01 | 0.04 | 0.01 | 14.77 | 5.50 | 0.78 |
| 0.01 | 0.01 | 0.01 | 0.34 | 0.31 | 1.00 | 0.00 | 0.00 | 0.00 | 0.98 | 0.82 | 0.57 |

¹⁷⁷Lu-PSMA-MT: 1 h

| | | | | | | | Small | | | | |
|---|---|---|---|---|---|---|---|---|---|---|---|
| Blood | Heart | Lung | Spleen | Liver | Kidney | Muscle | Intestine | Brain | Tumor | Kidney | Tail |
| 0.35 | 0.16 | 0.56 | 3.83 | 2.07 | 144.27 | 0.08 | 0.15 | 0.02 | 12.83 | 142.04 | 4.61 |
| 0.24 | 0.07 | 0.14 | 1.36 | 1.90 | 23.66 | 0.04 | 0.06 | 0.01 | 1.30 | 26.70 | 5.36 |

¹⁷⁷Lu-PSMA-MT: 4 h

| | | | | | | | Small | | | | |
|---|---|---|---|---|---|---|---|---|---|---|---|
| Blood | Heart | Lung | Spleen | Liver | Kidney | Muscle | Intestine | Brain | Tumor | Kidney | Tail |
| 0.04 | 0.04 | 0.10 | 3.94 | 4.58 | 20.59 | 0.02 | 0.04 | 0.01 | 9.18 | 19.45 | 0.50 |
| 0.02 | 0.01 | 0.03 | 0.25 | 0.30 | 12.68 | 0.01 | 0.02 | 0.00 | 1.58 | 10.01 | 0.12 |

### Example 7: Identification of the Alpha-Amylase Cleavage Site in PSMA-MT

10 μl of a solution of PSMA-MT in ultrapure water with a concentration of 1 mM were analyzed by analytic RP-HPLC and MALDI-TOF before the digestion. For digestion 10 μl of α-Amylase (1 U/μl in H2O ) were added to 10 μl of a solution of PSMA-MT in ultrapure water with a concentration of 1 mM and incubated at RT for 1 h. The solution was afterwards analyzed with analytic RP-HPLC and MALDI-TOF to evaluate the reaction.

In MALDI-TOF analysis a peak of the original mass of 1848.97 g/mol was detected, which disappeared after 1 h of digestion with α-Amylase, indicating a complete turnover of PSMA-MT.

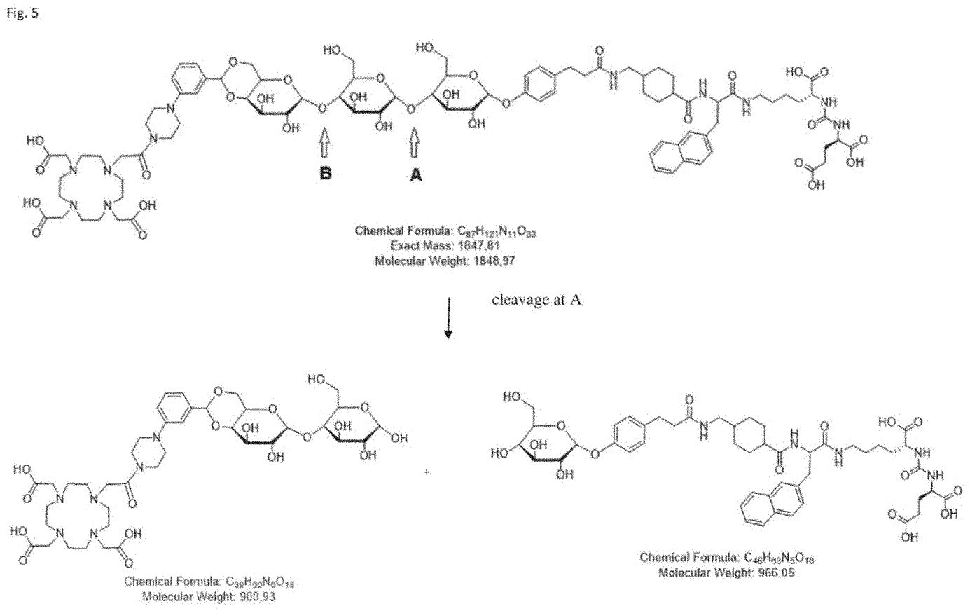

The potential cleavage sites of alpha-amylase in PSMA-MT are shown in FIG. 5, as are products of cleavage at cleavage position A.

To investigate the exact digest position of PSMA-MT the different fragments found in the MALDI-TOF after the digest were compared. Two main peaks occurred at 902 g/mol and at 989 g/mol. The first fragment with the DOTA-chelator C₃₅H₆₀N₆O₁₈+H⁺ (901+1 g/mol) and the second fragment with the PSMA-binding-motive C₄₆H₆₃N₅O₁₆+Na⁺ (966+23 g/mol) predicted for cleavage site A were found in MALDI-TOF. Fragments corresponding to cleavage at cleavage site B could not be detected in the MALDI-TOF.

1. PSMA binding ligand comprising an oligosaccharide building block which comprises a bond being cleavable by alpha-amylase.

2. PSMA binding ligand according to claim 1, further comprising a PSMA binding motif Q and a chelator residue A, wherein the PSMA binding motif Q and the chelator residue A are preferably linked via at least one linker L^{AQ} comprising the oligosaccharide building block, the PSMA binding ligand preferably having the structure (I)
A-L^{AQ}-Q (I),
or a pharmaceutically acceptable salt or solvate thereof.

3. PSMA binding ligand according to claim 1, having the structure (Ia) 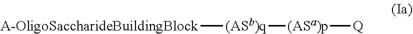
or a pharmaceutically acceptable salt or solvate thereof, wherein Q is the PSMA binding motif, A is the chelator residue, AS^{a} and AS^{b} are amino acid building blocks and q is an integer of from 0-4 and p is an integer of from 0 to 3.

4. PSMA binding ligand according to claim 1, wherein the oligosaccharide building block comprises of from 2 to 10, preferably of from 3 to 10, more preferably of from 3 to 6 monosaccharide units, most preferably 3 monosaccharide units.

5. PSMA binding ligand according to claim 1, comprising a PSMA binding motif Q and a chelator residue A, wherein the chelator residue A is derived from a chelator selected from the group consisting of 1,4,7,10-tetraazacyclododecane-N,N′,N″,N″′-tetraacetic acid (=DOTA), N,N″-bis[2-hydroxy-5-(carboxyethyl)benzyl]ethylenediamine-N,N″-diacetic acid, 1,4,7-triazacyclononane -1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid] (NOPO), 3,6,9,15-tetraazabicyclo[9.3.1.]pentadeca -1(15),11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N-hydroxysuccinamide (DFO), Diethylenetriaminepentaacetic acid (DTPA), trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl -DTPA (MX-DTPA).

6. PSMA binding ligand according to claim 5, wherein A is a chelator residue having a structure selected from the group consisting of

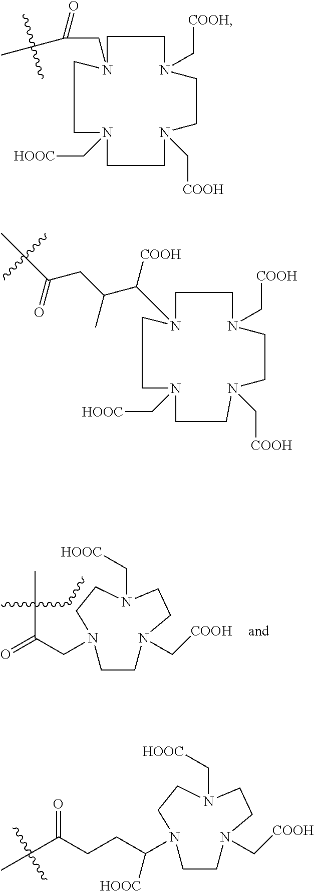

7. PSMA binding ligand according to claim 1, comprising a PSMA binding motif Q having the structure 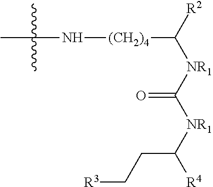
wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂.

8. PSMA binding ligand according to formula (Ia) 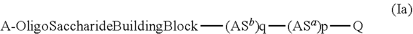
or a pharmaceutically acceptable salt or solvate thereof, wherein Q is a PSMA binding motif, A is a chelator residue, AS^{a} and AS^{b} are amino acid building blocks and q is an integer of from 0 to 4, wherein p is an integer of from 1 to 3.

9. PSMA binding ligand according to claim 8, wherein AS^{a} has the structure 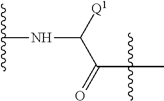
wherein Q¹ is selected from the group consisting of alkylaryl, arylalkyl, aryl, alkylheteroaryl, heteroarylalkyl and heteroaryl.

10. PSMA binding ligand according to claim 8, wherein AS^{b} has the structure (b) 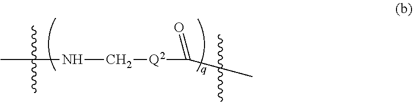
wherein Q² is selected from the group consisting of aryl, alkylaryl, arylalkyl, cycloalkyl, heterocycloalkyl, heteroaryl, heteroarylalkyl and alkylheteroaryl, and wherein q is an integer of from 0-4, preferably wherein Q² is 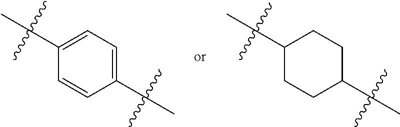 [SMILES: *c1ccc(*)cc1 ; *C1CCC(*)CC1]
preferably

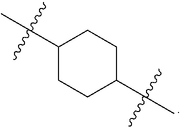 [SMILES: *C1CCC(*)CC1]

11. PSMA binding ligand according to claim 8, wherein A is a chelator residue derived from a chelator selected from the group consisting of 1,4,7,10-tetraazacyclododecane-N,N′,N″,N″′-tetraacetic acid (=DOTA), N,N″-bis[2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid] (NOPO), 3,6,9,15-tetraazabicyclo[9.3.1.]pentadeca -1(15),11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p -isothiocyanatobenzyl)-4-methyl-DTPA (1 M3B) and 1-(2)-methyl-4-isocyanatobenzyl -DTPA (MX-DTPA).

12. Complex comprising
(a) a radionuclide, and
(b) the PSMA binding ligand according to claim 1 or a pharmaceutically acceptable salt or solvate thereof.

13. The complex of claim 12, wherein, the radionuclide is selected from the group consisting ⁸⁹Zr, ⁴⁴_{Sc,} ¹¹¹ln, ⁹⁰Y, ⁶⁶Ga, ⁶⁷Ga, ⁶⁸Ga, ¹⁷⁷Lu, ^{99m}Tc, ⁶⁰Cu, ⁶¹Cu, ⁶²Cu, ⁶⁴Cu, ⁶⁶Cu, ⁶⁷Cu, 149Tb, ¹⁵²Tb, ¹⁵⁵Tb, ¹⁵³Sm, ¹⁶¹Tb, ¹⁵³Gd, ¹⁵⁵Gd, ¹⁵⁷Gd, ²¹³Bi, ²²⁵Ac, ²³⁰U, ²²³Ra, ¹⁶⁵Er, ⁵²Fe, ⁵⁹Fe, and radionuclides of Pb (such as ²⁰³Pb and ²¹²Pb, ²¹¹Pb, ²¹³Pb, 214Pb, ²⁰⁹Pb, ¹⁹⁸Pb, ¹⁹⁷Pb).

14. A pharmaceutical composition comprising the PSMA binding ligand of claim 1.

15. A method for treating and/or preventing prostate cancer and/or metastases thereof with the PSMA binding ligand of claim 1.

16. A method for diagnosing cancer, preferably prostate cancer and/or metastases thereof, with a PSMA binding ligand of claim 1.

17. PSMA binding ligand according to claim 9, wherein AS^{b} has the structure (b) 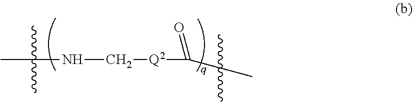
wherein Q² is selected from the group consisting of aryl, alkylaryl, arylalkyl, cycloalkyl, heterocycloalkyl, heteroaryl, heteroarylalkyl and alkylheteroaryl, and wherein q is an integer of from 0-4, preferably wherein Q² is 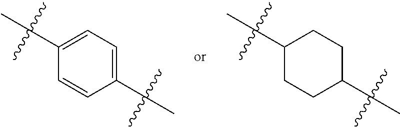 [SMILES: *c1ccc(*)cc1 ; *C1CCC(*)CC1]
preferably 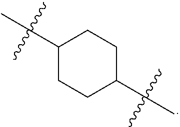 [SMILES: *C1CCC(*)CC1]
